(19) United States
(12) Patent Application Publication
(10) Pub. No.: US 2026/0194664 A1
(43) Pub. Date: Jul. 9, 2026

(54) MEDICAL IMAGING SYSTEM WITH DEPTH-OF-INTERACTION AND COMPTON SCATTER RESOLVING CAPACITIES AND RELATED METHOD

(71) Applicant: CANON MEDICAL SYSTEMS CORPORATION, Tochigi (JP)

(72) Inventors: Xiaoli LI, Vernon Hills, IL (US); Kent BURR, Vernon Hills, IL (US); Yi QIANG, Vernon Hills, IL (US); Peng PENG, Vernon Hills, IL (US)

(73) Assignee: CANON MEDICAL SYSTEMS CORPORATION, Tochigi (JP)

(21) Appl. No.: 19/014,366

(22) Filed: Jan. 9, 2025

(51) Int. Cl. G01T 1/164; A61B 6/42; G01T 1/29

(52) U.S. Cl. CPC G01T 1/1642; A61B 6/4258; G01T 1/2985

(57) ABSTRACT

A medical imaging apparatus based on gamma-ray detection includes a detector and circuitry. The detector includes a plurality of detector modules. Each detector module is divided into N light-sharing segments, and each light-sharing segments covers M photosensors. The circuitry includes (1) a plurality of sets of M first-level electronic units and (2) a second-level electronic unit. Each set of M first-level electronic units reads the electrical signals generated by of a corresponding one of the plurality of detector modules. For each detector module, each first-level electronic unit of a corresponding set of M first-level electronic units reads the electrical signals generated by N photosensors, in a multiplexing manner, to generate timing, energy, and position readout signals on a single set of readout channels. The second-level electronic unit processes the timing, energy, and position readout signals output from M sets of readout channels of the set of M first-level electronic units.

### CROSS-REFERENCE TO RELATED APPLICATIONS

This application is related to U.S. patent application Ser. No. 18/415,012 (Attorney Docket No. 550329US) entitled “METHOD AND APPARATUS FOR DETERMINING TIME OF FLIGHT AND DEPTH OF INTERACTION USING A POSITRON EMISSION TOMOGRAPHY SYSTEM”, filed on Jan. 17, 2024, the contents of which are incorporated herein by reference.

### BACKGROUND

### Field

This disclosure relates to medical imaging systems based on gamma-ray detection, including, but not limited to, positron emission tomography (PET) imaging systems, single-photon emission computed tomography (SPECT) imaging systems, etc.

### Description of the Related Art

Positron emission tomography (PET) is a functional imaging modality that is capable of imaging biochemical processes in humans or animals through the use of radioactive tracers. In PET imaging, a tracer agent is introduced into the patient to be imaged via injection, inhalation, or ingestion. After administration, the physical and bio-molecular properties of the agent cause it to concentrate at specific locations in the patient's body. The actual spatial distribution of the agent, the intensity of the region of accumulation of the agent, and the kinetics of the process from administration to its eventual elimination are all factors that may have clinical significance.

During this process, a tracer attached to the agent will emit positrons. When an emitted positron collides with an electron, an annihilation event occurs, wherein the positron and electron are combined. An annihilation event produces two gamma-ray photons (with an energy level of 511 keV) traveling at substantially 180 degrees apart.

There has been significant interest in depth-of-interaction (DOI) capable detectors for pre-clinical PET scanners and brain PET scanners, with the goal of achieving high spatial resolution in the reconstructed images. The demand for DOI-capable time-of-flight (TOF) detectors is growing in clinical whole-body scanners, particularly with the increased scanner axial field of view (FOV) and advancements in scanner spatial resolution and timing resolution.

The primary reason for incorporating DOI is to reduce parallax error for oblique lines of response (LOR). Historically the impact of parallax error in clinical whole-body imaging has been relatively small. The relative impact of parallax error is increasing, however, as spatial resolution is improved, with the use of smaller scintillator pixels, and axial FOV is increased, which generates more oblique LORs.

In PET imaging, it is possible to enhance the signal-to-noise ratio (SNR) of the images through improved timing resolution. To achieve sufficient sensitivity, LYSO scintillators that are commonly used in TOF PET scanners typically have a crystal thickness of about 20 mm. However, timing resolution tends to degrade as the crystal thickness increases.

Integrating DOI information has the potential to enhance timing resolution. As commercial PET scanners approach a timing resolution of approximately 200 ps, the integration of DOI information can potentially further enhance timing resolution through careful detector design and calibration.

Furthermore, when considering the gamma-photon-interaction physics in crystals, inter-crystal Compton scatter events may happen, leading to image blurring if the first interactions are not properly resolved. Inter-crystal Compton scatter events often consist of multiple interactions occurring at different depths of the crystals, which can make the DOI decoding more challenging. However, most existing methods use energy-weighted average position for multi-interaction events, and thus the inter-crystal Compton scatter events are not properly resolved.

It is desirable to develop a PET scanner equipped with both DOI and Compton scatter resolving capabilities, so as to enhance the overall performance of clinical PET scanners.

### SUMMARY

The present disclosure relates to a medical imaging apparatus based on gamma-ray detection. The medical imaging apparatus includes a detector and circuitry. The detector includes a plurality of detector modules, each detector module including a crystal array and a photosensor array coupled to the crystal array. The crystal array includes a plurality of scintillation crystals that generate scintillation light in response to gamma-ray interactions occurring therein. The photosensor array includes a plurality of photosensors that generate electrical signals upon detecting the scintillation light emitted from the crystal array. Each detector module is divided into N light-sharing segments, and each of the N light-sharing segments covers M photosensors of the photosensor array, where N and M are integers, N≥2, and M≥2. The circuitry includes (1) a plurality of sets of M first-level electronic units and (2) a second-level electronic unit. Each set of M first-level electronic units is configured to read the electrical signals generated by the photosensor array of a corresponding one of the plurality of detector modules. For each detector module of the plurality of detector modules, each first-level electronic unit of a corresponding set of M first-level electronic units reads the electrical signals generated by N photosensors, in a multiplexing manner, to generate timing, energy, and position readout signals on a single set of readout channels, the second-level electronic unit processes the timing, energy, and position readout signals output from M sets of readout channels of the set of M first-level electronic units, with respect to each first-level electronic unit of the set of M first-level electronic units, each of the N photosensors read by the first-level electronic unit is situated in a corresponding different one of the N light-sharing segments, and with respect to each light-sharing segment of the N light-sharing segments, each of the M photosensors covered by the light-sharing segment is read by a corresponding different first-level electronic unit of the set of M first-level electronic units.

The disclosure additionally relates to a method for reading and processing electric signals in a medical imaging apparatus based on gamma-ray detection. The medical imaging apparatus includes a detector and circuitry. The detector includes a plurality of detector modules, each detector module including a crystal array and a photosensor array coupled to the crystal array. The crystal array includes a plurality of scintillation crystals that generate scintillation light in response to gamma-ray interactions occurring therein. The photosensor array includes a plurality of photosensors that generate electrical signals upon detecting the scintillation light emitted from the crystal array. Each detector module is divided into N light-sharing segments, and each of the N light-sharing segments covers M photosensors of the photosensor array, where N and M are integers, N≥2, and M≥2. The circuitry includes (1) a plurality of sets of M first-level electronic units and (2) a second-level electronic unit. Each set of M electronic units is configured to read the electrical signals generated by the photosensor array of a corresponding one of the plurality of detector modules. The method includes, for each detector module of the plurality of detector modules: reading via each first-level electronic unit of a corresponding set of M first-level electronic units, the electrical signals generated by N photosensors in a multiplexing manner to generate timing, energy, and position readout signals on a single set of readout channels; and processing via the second-level electronic unit, the timing, energy, and position readout signals output from M sets of readout channels of the set of M first-level electronic units. With respect to each first-level electronic unit of the set of M first-level electronic units, each of the N photosensors read by the first-level electronic unit is situated in a corresponding different one of the N light-sharing segments, and with respect to each light-sharing segment of the N light-sharing segments, each of the M photosensors covered by the light-sharing segment is read by a corresponding different first-level electronic unit of the set of M first-level electronic units.

The disclosure additionally relates to a non-transitory computer readable medium having instructions stored therein that, when executed by one or more processors, cause the one or more processors to perform the above method for reading and processing electric signals in a medical imaging apparatus based on gamma-ray detection.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 shows a parallax error arising from an oblique line-of-response (LOR) in a pair of positron emission tomography (PET) detector pixels without depth of interaction (DOI) capacities;

FIG. 2A shows the structure of a miniblock used in a DOI PET detector for achieving light sharing, in accordance with embodiments of the present disclosure;

FIG. 2B shows transparency variation along the z direction (DOI direction) of an inner reflector within the miniblock, in accordance with embodiments of the present disclosure;

FIG. 3 shows exemplary gamma interactions occurring in a PET detector with a segmented light sharing design, in accordance with embodiments of the present disclosure;

FIGS. 4A, 4B, and 4C show three exemplary designs for decoding DOI information based on the amount of light sharing, in accordance with embodiments of the present disclosure;

FIG. 5 shows a scenario where DOI information is decoded from the ratio of the amount of light collected by a photosensor directly underneath a crystal versus the total amount of light collected by all photosensors within the same light-sharing segment, in accordance with embodiments of the disclosure;

FIG. 6 shows a set of digital output channels (T, E) for reading signals from a single photosensor;

FIG. 7 shows an exemplary first-level electronic unit with a single set of digital output channels (T, E, X, Y) for reading signals from N photosensors, in accordance with embodiments of the disclosure;

FIG. 8A shows the connection between a photosensor array and four first-level electronic units, in accordance with embodiments of the disclosure;

FIGS. 8B, 8C, and 8D show the E-, X-, and Y-multipliers (or weights) used in the Anger logic within the four first-level electronic units, in accordance with embodiments of the disclosure;

FIG. 9A shows a multiplexing scheme with the same numbers of photosensors and first-level electronic units as FIG. 8A;

FIGS. 9B, 9C, and 9D show the E-, X-, and Y-weights used in the Anger logic within the four first-level electronic units, in accordance with the multiplexing scheme of FIG. 9A;

FIGS. 10A, 10B, 10C, and 10D show exemplary weights and three sets of possible signal levels that can produce the same E, X, and Y measurements, in accordance with the multiplexing scheme of FIG. 9A;

FIG. 11 shows a flow chart of an exemplary procedure for determining energy, position, and timing information for an event, in accordance with embodiments of the disclosure;

FIG. 12 shows an exemplary flood histogram generated for the detector design shown in FIGS. 8A-8D, in accordance with embodiments of the present disclosure;

FIGS. 13A-13C show exemplary properties of single-light-sharing-segment events, four selected two-light-sharing-segment events, and two selected three-light-sharing-segment events, in accordance with embodiment of the present disclosure;

FIGS. 14A-14F show exemplary look-up tables (LUTs) generated for the detector design shown in FIGS. 8A-8D, in accordance with embodiments of the present disclosure;

FIG. 15 shows an example where three LUTs overlap to varying degrees;

FIG. 16 shows an exemplary event obtained from a simulated data set, with black dots representing the positions of detected optical photons at individual SiPMs;

FIG. 17 shows exemplary voting regions of different first-level electronic units for the interaction shown in FIG. 16;

FIG. 18A-F show, within the LUTs, the boundaries of the voting regions for the interaction shown in FIG. 16, where the numbers of votes casted for the respective LUTs are shown on the x-axis labels;

FIG. 19 shows voting regions of different first-level electronic units for another exemplary interaction;

FIG. 20A-20F show, within the LUTS, the boundaries of the voting regions as shown in FIG. 19, where the numbers of votes casted for the respective LUTs are shown on the x-axis labels;

FIG. 21 shows voting regions of different first-level electronic units for another exemplary interaction;

FIG. 22A-22F show, within the LUTS, the boundaries of the voting regions as shown in FIG. 21, where the numbers of votes casted for the respective LUTs are shown on the x-axis labels;

FIGS. 23A-23D illustrate a scenario using an additional tie-breaker to differentiate between overlapping “diagonal” two-light-sharing-segment interactions, in accordance with embodiments of the disclosure;

FIG. 24 shows voting regions of different first-level electronic units for another exemplary interaction;

FIG. 25A-25F show, within the LUTS, the boundaries of the voting regions as shown in FIG. 24, where the numbers of votes casted for the respective LUTs are shown on the x-axis labels;

FIG. 26 illustrates two three-light-sharing-segment LUTs (in gray) that overlap and four two-light-sharing-segment LUTs (in black) that are relevant to those three-light-sharing-segment LUTs;

FIG. 27A-27D show a scenario using a tie-breaker voting to differentiate between the two overlapping three-light-sharing-segment LUTs shown in FIG. 26, in accordance with embodiments of the present disclosure;

FIG. 28 shows an exemplary structure of a single neutral network that infers the light-sharing segment number(s) (ID(s)) of the light-sharing segment(s) involved in an event and signal levels at each SiPM within the involved light-sharing segment(s), in accordance with embodiments of the present disclosure;

FIG. 29 shows a high-level view of the architecture of a multi-stage neural network that infers the light-sharing segment number(s) (ID(s)) of the light-sharing segment(s) involved in an event and signal levels at each SiPM within the involved light-sharing segment(s), in accordance with embodiments of the present disclosure;

FIG. 30 shows an exemplary structure of the first stage of the multi-stage neural network shown in FIG. 29, in accordance with embodiments of the present disclosure;

FIG. 31 shows an exemplary structure of the second stage of the multi-stage neural network shown in FIG. 29, in accordance with embodiments of the present disclosure;

FIG. 32A shows an exemplary structure of the third stage of the multi-stage neural network shown in FIG. 29, in accordance with embodiments of the present disclosure;

FIG. 32B shows an exemplary structure of the third stage of the multi-stage neural network shown in FIG. 29, in accordance with embodiments of the present disclosure;

FIG. 32C shows an exemplary structure of the third stage of the multi-stage neural network shown in FIG. 29, in accordance with embodiments of the present disclosure;

FIG. 33 shows a high-level view of the architecture of a neural network for estimating the final energy, position and time stamp of a gamma event, in accordance with embodiments of the present disclosure;

FIG. 34A shows an exemplary DOI detector design with segmented light sharing achieved through a light guide, in accordance with embodiments of the present disclosure;

FIG. 34B shows an exemplary DOI detector design with segmented light sharing achieved through partial reflectors and partial optical glue, in accordance with embodiments of the present disclosure;

FIG. 35 shows the connection between a photosensor array and nine first-level electronic units, in accordance with embodiments of the present disclosure;

FIG. 36A shows a perspective view of a PET scanner that can be used with the techniques described herein; and

FIG. 36B shows a schematic view of a PET scanner that can be used with the techniques described herein.

### DETAILED DESCRIPTION

The following disclosure provides embodiments or examples for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,” unless stated otherwise.

To achieve high sensitivity, positron emission tomography (PET) imaging scanners typically use thick detectors to give the required 511 keV stopping power. However, uncertainty in depth of interaction (DOI) information within these thick detectors can result in parallax errors. Such parallax degradation can become even worse with increasing radial position from the center of the PET field of view.

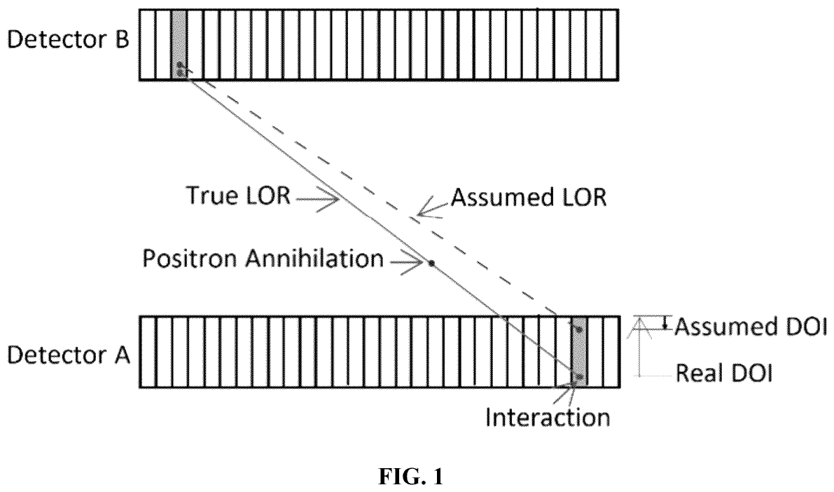

FIG. 1 shows a scenario where a parallax error arises from an oblique line-of-response (LOR) in PET detector pixels that lack DOI information. Without DOI capacities, gamma-ray interaction events, which can occur over all depths within a detector pixel, are attributed to a single position. For example, the dashed line in FIG. 1 represents the assumed LOR, which is the same for all coincidence events between those two detector pixels. The solid line represents the true LOR, which can be exactly drawn if DOI information for that specific coincidence event is available. The parallax errors caused can lead to artifacts and degradation in image quality.

Various methods have been developed to implement DOI encoding detectors. These methods include the phoswich detector design, the layered-crystals-with-offset-structure design, the layered-crystals-with-mixed-shape design, the multiple crystal-photosensor layers design, and the dual-ended photosensors design, etc. However, each of these designs has its own drawbacks, especially when applied in clinical PET scanners.

For instance, in the phoswich detector design, each scintillator is composed of two or more different materials with measurable differences in their scintillation decay times to encode DOI information. The differences between the scintillation materials can degrade timing or energy performance of the detector.

In the layered-crystals-with-offset-structure design, despite using the same crystal material, there is an offset in crystal pitches between different layers to encode DOI information. This offset structure, by adding optical interfaces where light can be reflected, increases the path length of scintillation photons, leading to degradation in timing and energy performance.

Similarly, the layered-crystals-with-mixed-shape design uses the same crystal material, but crystal shapes vary in different layers to encode DOI information. This mixed shape structure also increases the path length of scintillation photons, resulting in degradation in timing and energy performance. Due to degraded timing performance, none of the above three designs is suitable for time-of-flight (TOF) detectors.

The multiple crystal-photosensor layers design splits scintillation elements (i.e., crystals) into two or more separate layers, each coupled individually to its photosensor and readout electronics. However, an increased number of photosensors and circuit boards and requirements for thin circuit boards between crystal-photosensor layers, results in high manufacturing costs.

In the dual-ended photosensors design, the crystal array is readout on both ends by two photosensor arrays, using the ratio of the total energy measured by the two readouts to encode DOI information. However, the manufacture cost is high with twice the number of photosensors and readout channels. Due to increased scanner costs from additional photosensors and electronic readout channels, the above two designs are too expensive for clinical PET scanners.

Apart from those designs, several methods utilize light sharing to implement DOI encoding, such as the DOI-detector-with-uniform-light-guide design, the DOI-detector-with-prism-light-guide design, and the DOI-detector-with-laser-engraved-subsurface design. These light sharing approaches encode DOI information by varying the amount of light sharing for events occurring at different depth of the crystal. Since no layered crystals are used, good optical photons transportation can be maintained and these designs do not degrade timing performance significantly when DOI information is properly included in the timing corrections that are always necessary in TOF PET systems. Additionally, compared with standard TOF detectors, the single-ended photosensor readout used in these designs does not increase scanner costs.

In the DOI-detector-with-uniform-light-guide design, the side of the scintillator array opposite to the photosensors is coupled to a light guide made of glass, for example. The light distribution among different photosensors is used to encode DOI information. This single-ended photosensors design reduces costs compared with dual-ended photosensors design. However, reading timing and energy for each photosensor channel can still be costly for a clinical PET scanner.

In the DOI-detector-with-prism-light-guide design, the side of the scintillator array opposite to the photosensors is coupled to a prism light guide array. The light distribution among different photosensors is used to encode DOI information. Similar to the DOI-detector-with-uniform-light-guide design, timing and energy are readout for each photosensor channel. Due to the specialized prism light guide array and the massive number of signal channels, the manufacture cost is likely to be too high for clinical PET scanners.

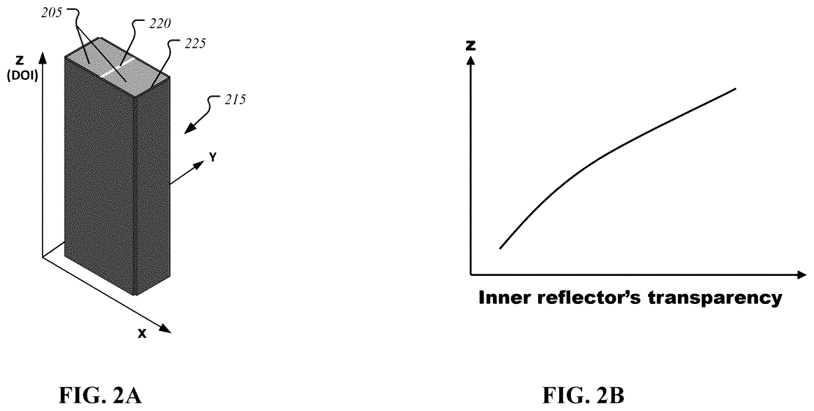

Taking U.S. patent application Ser. No. 18/415,012 (Attorney Docket No. 550329US) as an illustrative instance of the DOI detector with depth-dependent transparency design, crystals within the PET detector are grouped together to form miniblocks. In a specific embodiment shown in FIG. 2A, each miniblock 215 has two crystals 205, and includes two different kinds of reflectors, i.e., an inner reflector 220 and outer reflectors 225. The inner reflector 220 separates the crystals 205 within the miniblock 215, exhibiting varying transparency along the z direction, i.e., the DOI direction, as shown in FIG. 2B.

For example, the z-dependent transparency can be achieved via a laser engraving process to create different patterns of openings or holes on or through the inner reflector 220. Alternatively, different materials along the z direction or varying thicknesses of the inner reflector 220 made from the same material can realize the desired z-dependent transparency. For instance, the inner reflector 220 can be formed using a light-diffusive reflective material (such as barium sulfate or titanium dioxide powder) with varying thicknesses in the z direction. This approach allows the encoding of DOI information with a longitudinally varying amount of light sharing in the miniblock 215.

The outer reflectors 225 separate the miniblocks 215 in the PET detector 200, so as to prevent light cross talk between those miniblocks 215. This spatial confinement of scintillation light within individual miniblocks 215 can ensure that a high concentration of scintillation light reaches the photosensors (not shown), contributing to higher timing resolution for time-of-flight measurements in the PET system.

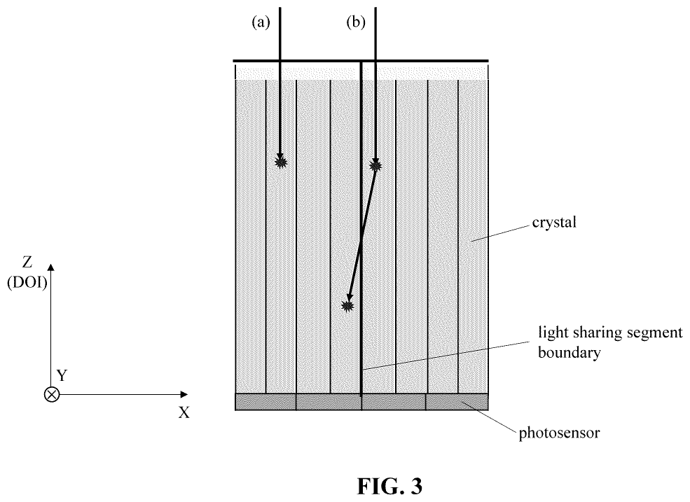

As mentioned previously, most existing methods have challenges in properly resolving the inter-crystal Compton scatter events. FIG. 3 shows exemplary gamma interactions in scintillation crystals. A standard photoelectric interaction, where the entire 511 keV energy is absorbed in one interaction, is illustrated at (a). In addition, as illustrated at (b), a Compton scatter interaction may occur, where the 511 keV energy is absorbed in two or more interactions, such as at least one Compton scatter interaction followed by a photoelectric interaction. Similarly, K X-rays (characteristic X-rays) are often emitted following Compton and/or photoelectric interactions. These emitted X-rays can also travel to and deposit energy in other scintillation crystals. Since K X-rays are limited to energies below about 65 keV, they tend to interact in the crystal in which they were emitted or in nearest-neighbor crystals. In this context, since both Compton and K X-rays can result in energy being deposited in two or more discrete locations, they can be considered analogous phenomena.

Furthermore, in the context of PET detectors using segmented light sharing designs (such as those achieved be through partial reflectors between crystals, a light guide on the top of crystals, or other light sharing techniques), scintillation light photons generated in one crystal can propagate to other crystals within the same light-sharing segment. It has been observed that a substantial majority (more than 99% of cases) of the gamma interactions involve one, two, or three light-sharing segments. Specifically, for one exemplary detector design detailed in this disclosure, in 75% of cases, the 511 keV energy is deposited in a single light-sharing segment (e.g., the event represented by (a) in FIG. 3), while in about 23% of cases, the 511 keV energy is deposited in two neighboring light-sharing segments (e.g., the event represented by (b) in FIG. 3).

The present disclosure provides a low-cost, modular, and scalable TOF PET detector with DOI decoding capability and Compton scatter resolving capability. A multiplexing scheme is implemented to reduce the number of required read-out channels and determine the signal levels at the individual photosensors. Therefore, this detector design not only enhances scanner performance, but also effectively controls manufacturing costs.

According to embodiments of the disclosure, an array of crystals is coupled to an array of photosensors. Each crystal can be coupled to one photosensor. Alternatively, a mini array of crystals can be coupled to group of photosensors. Light sharing is implemented between a group of crystals to encode 3-D information through the amount of light sharing between different photosensors that are coupled to the light-sharing segment. The encoded 3-D information includes information about which crystal was hit and DOI information.

The segmented light sharing can be implemented through various designs, including, but not limited to, the approaches described in U.S. patent application Ser. No. 18/415,012 (Attorney Docket No. 550329US). For example, the detector can use scintillation crystals processed via subsurface laser engraving to generate point-like defects within the crystal. The pattern of these defects enables encoding of DOI information with a longitudinally varying amount of light sharing.

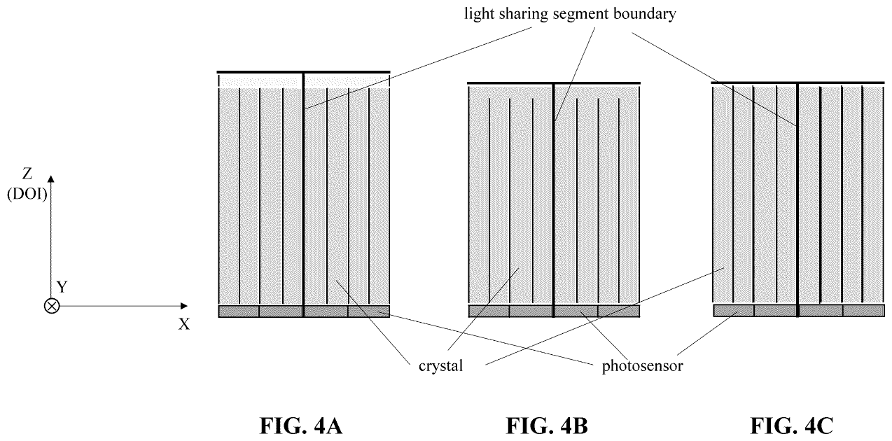

Three exemplary light sharing designs are shown in FIGS. 4A, 4B, and 4C. In FIG. 4A, light sharing is achieved through a uniform light guide arranged on the side of the crystal array opposite to the photosensors. In FIG. 4B, light sharing is realized through partial reflectors and partial optical glue applied between crystals within a light-sharing segment. In FIG. 4C, light sharing is achieved using triangular reflectors positioned between crystals within a light-sharing segment. Note that these light sharing designs are illustrative, and those skilled in the art can appreciate that various other designs can be implemented without departing from the concept and scope of this disclosure.

For instance, in the exemplary light sharing design shown in FIG. 4A, the four lateral surfaces of each crystal can be depolished, while the two end surfaces can be either polished or depolished, so as to enhance the DOI encoding performance.

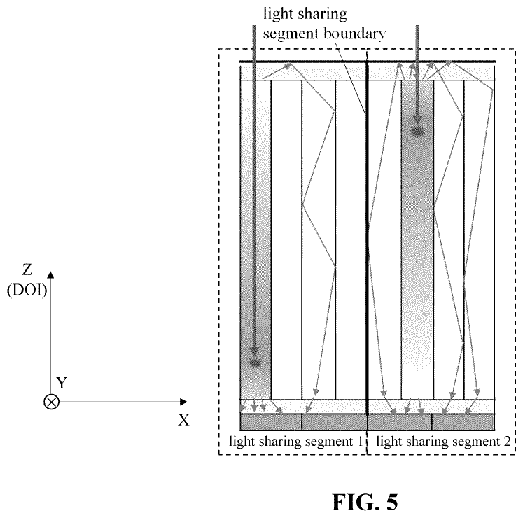

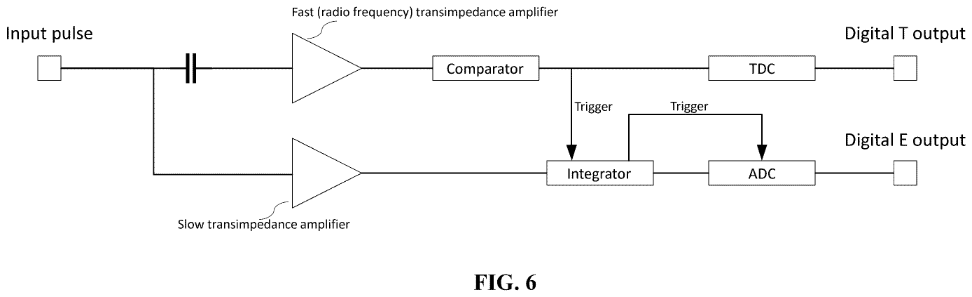

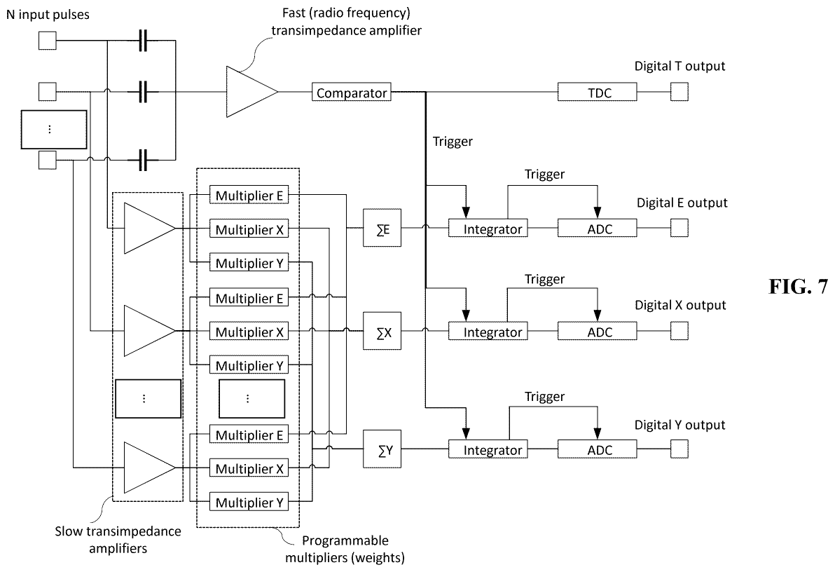

As further shown in FIG. 5, in each of the light-sharing segments 1 and 2, the light going to the bottom surface of a crystal is primarily detected by the photosensor directly underneath that crystal. In contrast, the light going to the top surface of the crystal can traverse through neighboring crystals within the light-sharing segment and be detected by photosensors underneath those crystals. In addition, the ratio of the amount of light going to the two end surfaces is dependent on the interaction depth along the crystal. As a result, the DOI information of an event can be decoded from the ratio of the amount of light collected by the photosensor directly underneath the crystal versus the total amount of light collected by all photosensors within the light-sharing segment.

Ideally, different light-sharing segments are optically isolated from one another. In practical scenarios where perfect isolation is not achieved, an initial estimate of signal levels on each photosensors can be made. Subsequently, from the signal pattern detected on the photosensors, a correction for optical crosstalk between the light-sharing segments can be implemented. This correction can obtain an improved estimate of the signal level on each photosensor.

In the example shown in FIG. 5, each 2×2 crystal array is coupled directly to one photosensor, and each light-sharing segment includes a 2×2 photosensor array. Therefore, one event which produces an interaction within a single light-sharing segment can generate four signals on photosensors associated with that light-sharing segment. Let Tᵢ, and Eᵢ represent the arrival time and energy of these signals, where i=1, 2, 3, 4. An energy ratio R, which encodes the DOI information, can be calculated as:

$$R = max ( {E}_{i} ) / sum ( {E}_{i} ) \qquad ( 1 )$$

FIG. 6 shows an exemplary set of signal read-out channels for one photosensor, where the digital “T” output represents the arrival time, and the digital “E” output represents the total signal level. Without a multiplexing readout scheme, this set of output channels needs to be deployed for each photosensor, leading to a massive number of signal read-out channels and high scanner costs. In contrast, a multiplexing approach with a high multiplexing ratio can preserve essential event information necessary for good scanner performance, while minimizing the load on the subsequent data acquisition (DAQ) system.

The present disclosure provides a signal multiplexing approach that can reduce the number of channels required for reading out signals from the photosensors, thereby mitigating PET scanner manufacturing costs. This multiplexing approach uses two levels of electronic units. Specifically, the first-level electronic unit functions as a multiplexing circuit for reading out a plurality of photosensors. The second-level electronic unit performs data processing for a plurality of first-level electronic units, facilitating the extraction of event information from the photosensors during both the calibration procedure and scanner operations.

FIG. 7 shows an exemplary first-level electronic unit in accordance with embodiments of the disclosure. As shown in FIG. 7, the fast outputs from N photosensors are readout, summed, and processed through a comparator to generate event triggers and timing pulses. Then, the timing pulses go through a time-to-digital converter (TDC) to derive digital timing outputs. The slow outputs from the N photosensors are readout, shaped, and transmitted through an Anger logic to generate energy and position pulses. The energy and position pulses then go through analog-to-digital converters (ADCs) to derive digital energy and position outputs E, X, and Y. During subsequent processing, the position of an event can be determined using these digital energy and position outputs, expressed as X′=X/E, and Y′=Y/E.

In the multiplexing approach shown in FIG. 7, the first-level electronic unit receives N input pulses from N photosensors, achieving a multiplexing ratio of N. As N photosensors can be efficiently read out by a single set of electronic channels T, E, X, and Y, the overall number of electronic channels can be reduced, leading to minimized scanner manufacturing costs and decreased power consumption. By selecting the number of photosensors coupled to one first-level electronic unit, various input multiplexing ratios can be achieved.

Moreover, the input pulses are acquired from N photosensors located in N different light-sharing segments. Let M denote the number of photosensors within each light-sharing segment. Each of the M photosensors within a single light-sharing segment is connected to one of M first-level electronic units. A second-level electronic unit covers these M first-level electronic units and N light-sharing segments.

In the first-level electronic unit shown in FIG. 7, upon the generation of an event trigger, a hit with timing, energy, and position outputs (T, E, X, Y) is generated. An event within a single light-sharing segment can generate M hits with separate timing, energy, and position outputs (Tᵢ, Eᵢ, Xᵢ, Yᵢ) in the M first-level electronic units, where i=I, II, III, IV, . . . , M. The final timing, energy, and position information of the event can be determined based on these M sets of timing, energy, and position outputs (Tᵢ, Eᵢ, Xᵢ, Yᵢ).

Further details of the multiplexing approach will be further described in the following exemplary detector designs.

### Detector Design 1

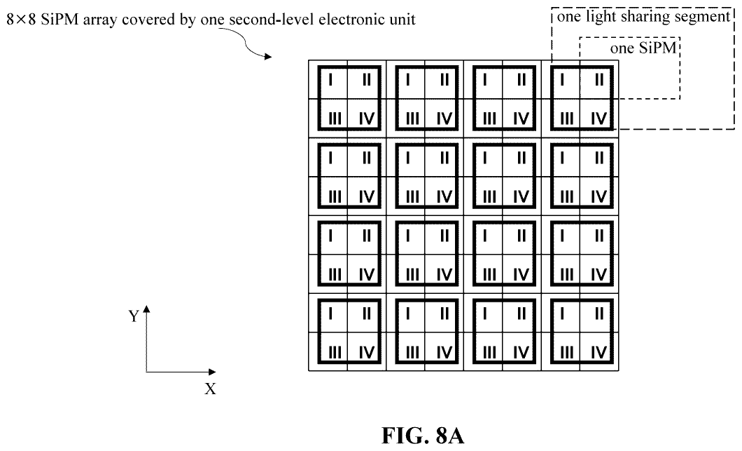

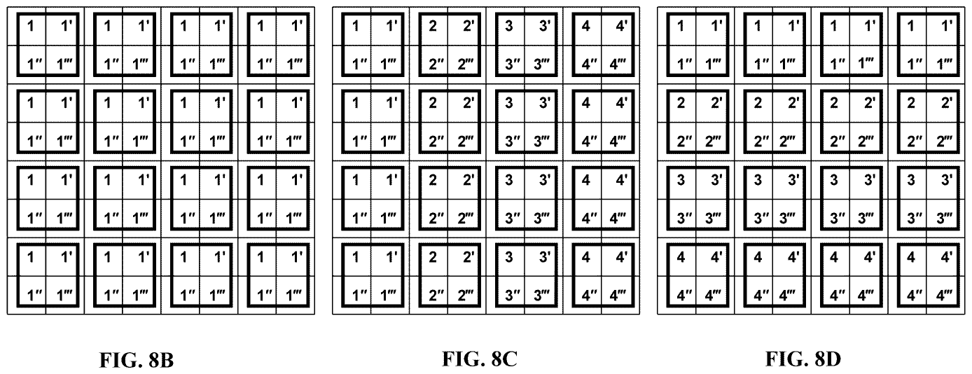

In this exemplary DOI TOF detector design, each detector module includes a 16×16 crystal array coupled to an 8×8 photosensor array. That is, each 2×2 crystal array is coupled directly to a single photosensor. The 8×8 photosensor array is covered by four first-level electronic units and one second-level electronic unit. Segmented light sharing is implemented within 2×2 photosensor arrays, forming 16 light-sharing segments. Using a commonly available single SiPM size of 4 mm×4 mm and the array sizes above results in single scintillator pixels of 2 mm×2 mm cross-sectional dimensions and light-sharing segments with 8 mm×8 mm cross-sectional area. An LYSO scintillator thickness (in the longitudinal dimension) of 20 mm can be chosen, although the scintillator thickness can be varied for different sensitivity and cost trade-offs. Similarly, a different scintillator material can be chosen for different performance and cost trade-offs. While SiPMs are shown as the photosensors in FIG. 8A and other drawings, one skilled in the art can recognize that the present disclosure is applicable to various other types of photosensors.

FIG. 8A illustrates the connection between the photosensors and the first-level electronic units I, II, III, and IV. The four photosensors within each 2×2 light-sharing segment are connected to the four first-level electronic units in a one-to-one manner. Consequently, each first-level electronic unit receives outputs from 16 photosensors located in 16 different light-sharing segments, resulting in an input multiplexing ratio of 16.

As previously mentioned, the first-level electronic units generate timing, energy, and position pulses through an Anger logic. The E-, X-, and Y-multipliers (or weights) used in the Anger logic are shown in FIGS. 8B, 8C, and 8D, respectively. In these figures, unmarked numbers indicate the E-, X-, and Y-weights applied to the first-level electronic unit I, those with ′ marks indicate the E-, X-, and Y-weights for the first-level electronic unit II, those with ″ marks indicate the E-, X-, and Y-weights for the first-level electronic unit III, and those with ″ marks indicate the E-, X-, and Y-weights applied to the first-level electronic unit IV.

In this exemplary design, segmented light sharing can be achieved using segmented glass tiles, segmented acrylic tiles, patterned light sharing/reflectors between crystals, for example. As discussed earlier, the individual light-sharing segments can be substantially isolated from each other. Within an individual light-sharing segment, the amount of light sharing is high enough so that every gamma ray which deposits 511 keV in the detector will trigger all four first-level electronic units in coincidence (i.e., within a short period of time, such as <1 ns). As described below, the coincident data from the four first-level electronic unit will be processed together.

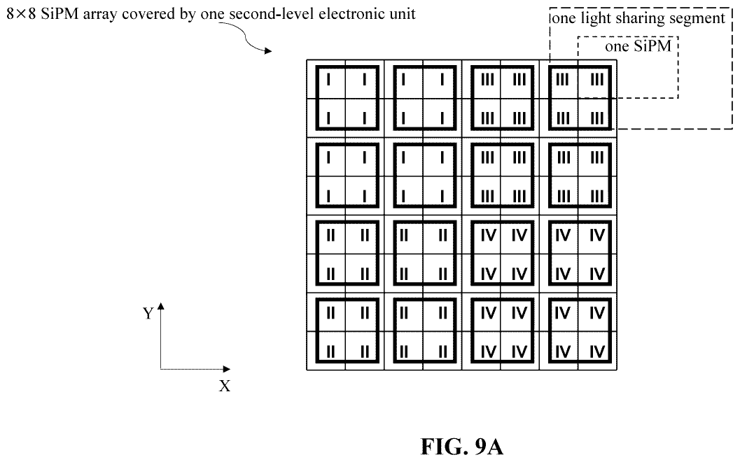

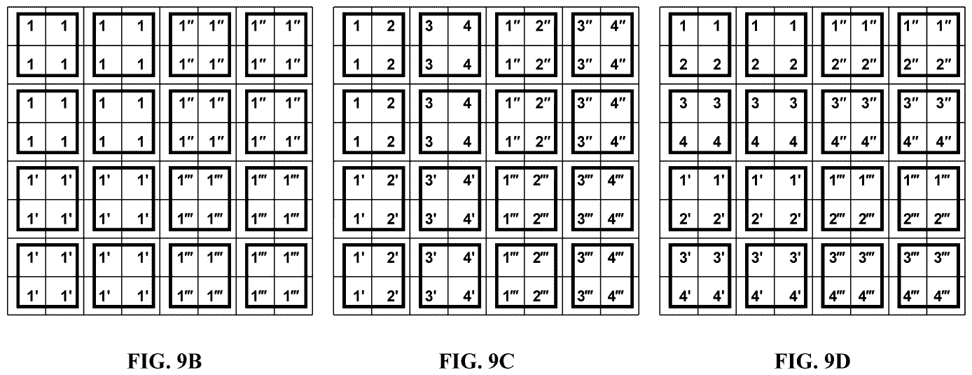

For comparison, FIG. 9A illustrates a multiplexing scheme with the same numbers of photosensors and first-level electronic units. The E-, X-, and Y-weights used in the Anger logic are shown in FIGS. 9B, 9C, and 9D, respectively.

In this multiplexing scheme, each light-sharing segment covers a 2×2 photosensor array within the detector module, while the photosensors in each quadrant of the detector module are connected to one of four first-level electronic units I, II, III, and IV. As a total of 16 photosensors is coupled to a single first-level electronic unit, the multiplexing ratio is 16 as well.

However, the multiplexing configuration shown in FIGS. 8A-8D can provide much more information than the one shown in FIGS. 9A-9D. Consider an event that deposits all its energy in a single light-sharing segment. In the multiplexing scheme of FIGS. 9A-9D, one first-level electronic unit will be triggered, resulting in a total of three measurements (E, X, Y). In contrast, using the multiplexing scheme of FIGS. 8A-8D, the event will trigger four first-level electronic units, resulting in 12 measurements (Eᵢ, Xᵢ, Yᵢ), where i=1, 2, 3, and 4. With only three measurements in the FIGS. 9A-9D scheme, it is impossible to determine the signal level on each of the four photosensors that belong to the same light-sharing segment.

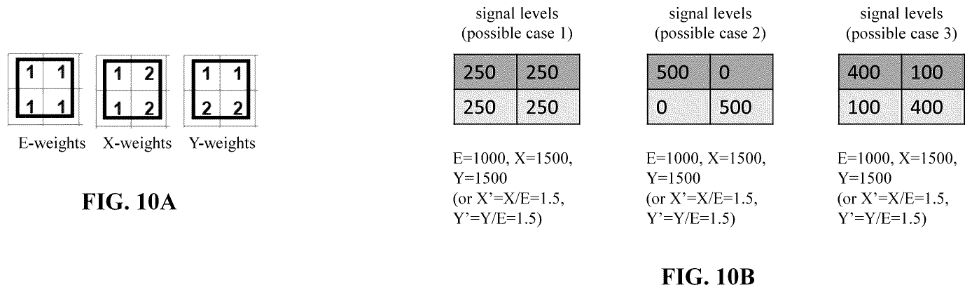

For example, when all photosensors within a light-sharing segment are connected to the same first-level electronic unit and the weights shown in FIG. 10A are used, three sets of possible signal levels (in arbitrary units) on the four photosensors can produce the same measurements E=1000, X=1500, and Y=1500, as shown in FIG. 10B. Since it is impossible to solve for four signal levels from three measurements, the multiplexing scheme of FIGS. 9A-9D loses information that is inherently available in the signals from the photosensors.

In contrast, the additional measurements provided by the FIGS. 8A-8D multiplexing scheme enable the determination of signal levels on each photosensor. In this scheme, one event results in 12 measurements. This means that for an event that deposits energy in one light-sharing segment (four unknown signal levels to solve for), two light-sharing segments (eight unknown signal levels), or three light-sharing segments (12 unknown signal levels), the signal level on each photosensor in the light-sharing segment(s) within which energy was deposited during the event can be resolved.

As mentioned previously, it has been observed that in this exemplary detector design, during roughly 25% of all events, the 511 keV energy is deposited in two or more light-sharing segments. The extra information provided by the multiplexing configuration shown in FIGS. 8A-8D can be used to accurately obtain 3-D position estimates (X″, Y″, and DOI) for interactions occurring in these light-sharing segments, calculating the energy, position, and timing information of these events. This facilitates the estimation of which hit should be considered the “first hit,” thereby providing improved quantitative image reconstruction.

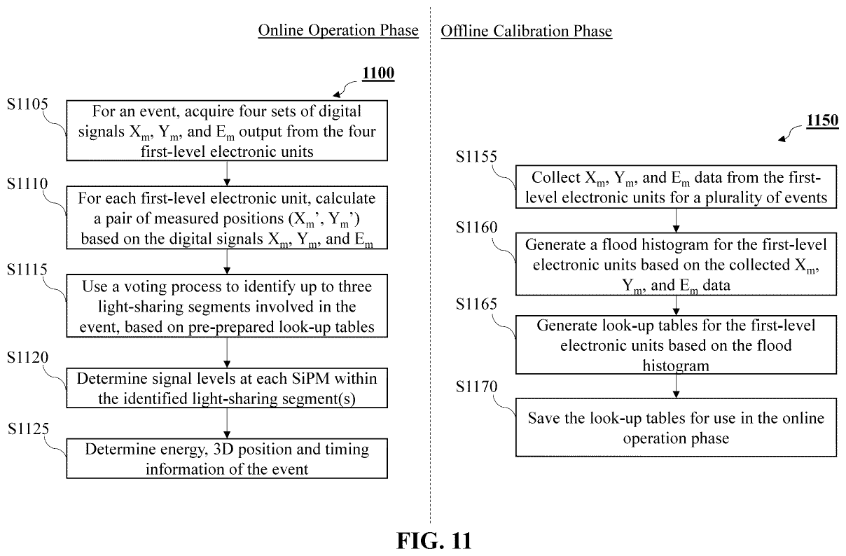

In this Detector Design 1, a single second-level electronic unit is used to process digital output signals from the four first-level electronic units. FIG. 11 shows an exemplary process executed by the second-level electronic unit to determine energy, position, and timing information for an event, in accordance with embodiments of the disclosure.

The process includes an online operation phase 1100 and an offline calibration phase 1150. The online operation phase 1100 begins at step S1105 by acquiring, with respect to the event, four sets of digital signals Xₘ, Yₘ, and Eₘ output from the four first-level electronic units, where m=I, II, III, and IV. In step S1110, a pair of measured positions (Xₘ′, Yₘ′) can be calculated for each first-level electronic unit, where Xₘ′=Xₘ/Eₘ, and Yₘ′=Yₘ/Eₘ. In step S1115, through a voting process based on the calculated (Xₘ′, Yₘ′) and a group of pre-prepared look-up tables, one, two, or three light-sharing segments involved in the event are identified. In step S1120, the signal levels at each SiPM within the identified light-sharing segment(s) are determined. In step S1125, energy, 3D position, and timing information of the event can be determined based on the determined signal levels at the SiPMs. The determined energy, 3D position and timing information can be used in subsequent image reconstruction.

The offline calibration phase 1150 prepares the look-up tables for use during the online operation phase 1100. In step S1155, Xₘ, Yₘ, and Eₘ data is collected from the first-level electronic units for a large number of events. In stepS1160, a flood histogram is generated for the first-level electronic units based on the collected Xₘ, Yₘ, and Eₘ data. In step S1165, look-up tables are generated for the first-level electronic units based on the flood histogram. In step S1170, the generated look-up tables are saved to be used during the online operation phase 1100.

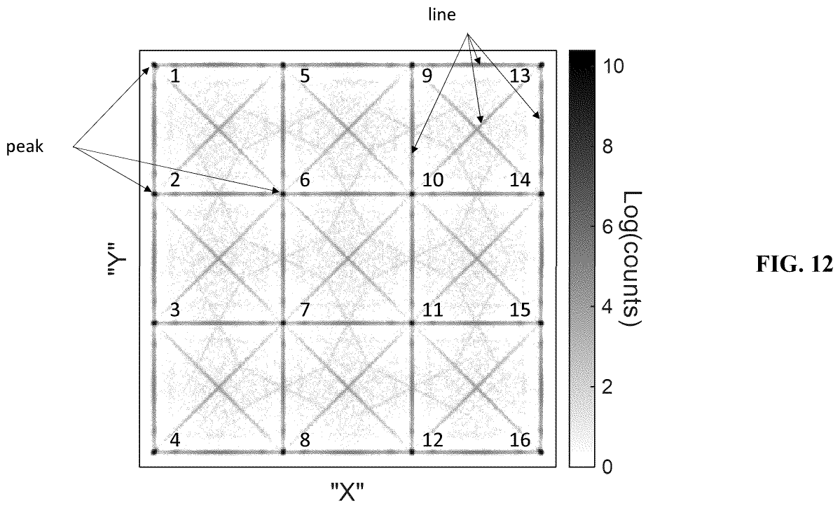

Further details of the online operation phase 1100 and the offline calibration phase 1150 can be found in the descriptions below, with reference to FIGS. 12-32C.

FIG. 12shows an exemplary flood histogram generated for the detector design shown in FIGS. 8A-8D, in accordance with embodiments of the present disclosure. Flood histograms, also known as “position maps,” represent 2-D histograms of “X” and “Y” positions for a large number of gamma-detection events detected by a single first-level electronic unit. Generally, the flood histograms generated for the four first-level electronic units appear to be very similar. The following description assumes that the four first-level electronic units share an identical flood histogram shown in FIG. 12. However, some variations may occur due to factors such as differences in amplifier gains and multiplier values, which are inherent to the electronics manufacturing process. As a result, individual flood histograms can be produced for each of the four first-level electronic units.

In the flood histogram of FIG. 12, each of the 16 “peaks” is formed by events depositing all of their energy in a single light-sharing segment, while each of the “lines” between two peaks is formed by events depositing energy in two neighboring light-sharing segments, typically due to a combination of Compton scatter and photoelectric interactions.

For instance, in FIG. 12, peaks #1-#16 are formed by events depositing all of their energy in a respective one of the light-sharing segments #1-#16. The lines connecting the two peaks 9 and 10, 9 and 13, 10 and 13, and 13 and 14 are formed by events splitting their energy in the light-sharing segments 9 and 10, 9 and 13, 10 and 13, and 13 and 14, respectively.

Let (xpeak_{m,n}, ypeak_{m,n}) denote the peak position for the n-th light-sharing segment as determined from the flood histogram generated for the m-th first-level electronic unit, where m and n are integers, I≤m≤IV, and 1≤n≤16. When an event involves interactions within one or more light-sharing segments, the measured positions (Xₘ′, Yₘ′) obtained for the m-th first-level electric unit are generated by a weighted sum of the interactions in each of the light-sharing segments (i.e., each of the “hit” segments) in which energy is deposited:

$${X}_{m}^{′} = {\sum }_{n = each hit segment} {r}_{m , n} {xpeak}_{m , n} \qquad ( 2 )$$

$$and$$

$$\tilde{{Y}_{m}^{′}} = {\sum }_{n = each hit segment} {r}_{m , n} {ypeak}_{m , n} \qquad ( 3 )$$

where r_{m,n} is the fraction of the total signal reaching the n-th light-sharing segment in the m-th first-level electronic unit. For each of the four first-electronic units, the fractional values sum to unity, i.e.,

$${\sum }_{n = each hit segment} {r}_{m , n} = 1 \qquad ( 4 )$$

Identifying the light-sharing segments involved in an event can be challenging. As shown in FIG. 12, two-segment interactions appear as lines between the single-segment peaks in the flood histogram. The lines between nearest-neighboring peaks are clearly visible. However, when a two-segment interaction involves more distantly separated light-sharing segments, the resulting line between non-nearest-neighboring peaks can be faint. Additionally, three-segment interactions can appear within triangular regions whose vertices are defined by three single-segment peaks corresponding to the segments involved. The low frequency of three-segment interactions and the overlapping nature of different combinations of these interactions make the visual identification of these regions quite difficult.

To tackle this problem, embodiments of the disclosure use look-up tables (LUTs) to describe the regions in which different interactions appear. For example, based on a flood histogram generated for the m-th first-level electronic unit, a separate LUT can be generated for each possible combination of light-sharing segments involved in an event. The LUT has binary values of 0 and 1, and is indexed by the measured position pair (Xₘ′, Yₘ′). Specifically, for a particular pair of (Xₘ′, Yₘ′), if the light-sharing-segment combination corresponding to a LUT (i.e., a linear combination of the peak positions of the light-sharing segments corresponding to the LUT) can produce that measured position pair (Xₘ′, Yₘ′), the LUT has a value of 1. In contrast, if the light-sharing-segment combination corresponding to a LUT cannot produce the measured pair (Xₘ′, Yₘ′), the LUT has a value of 0.

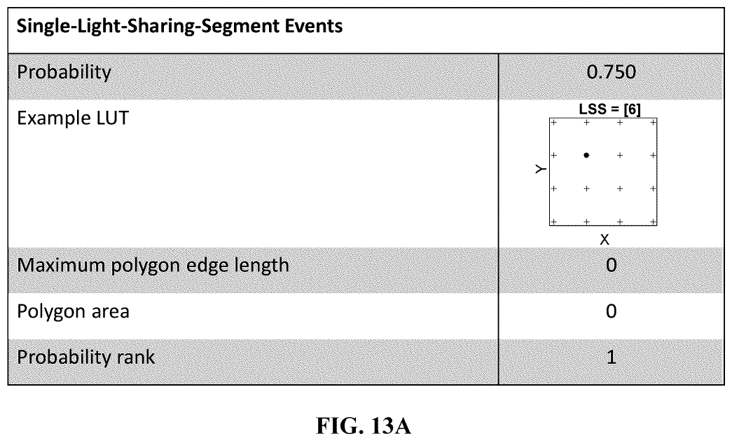

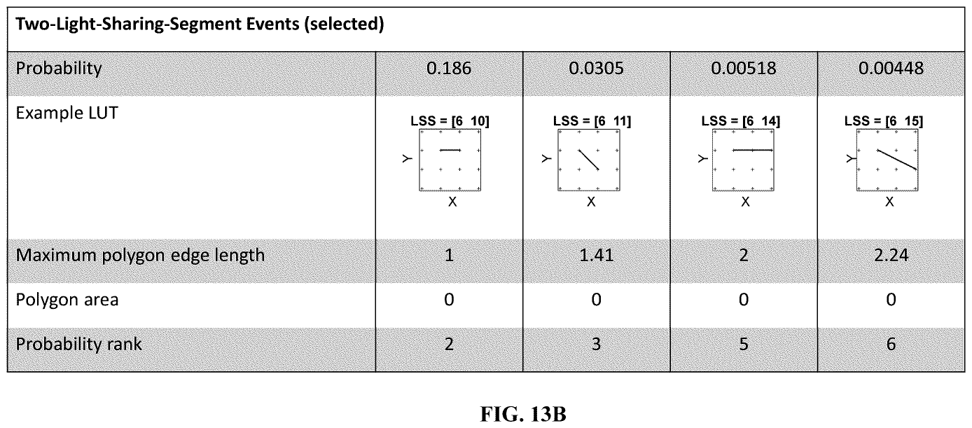

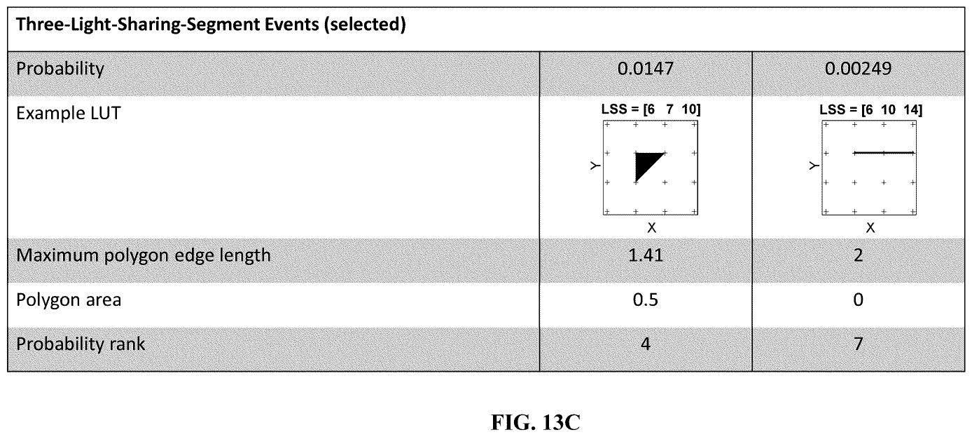

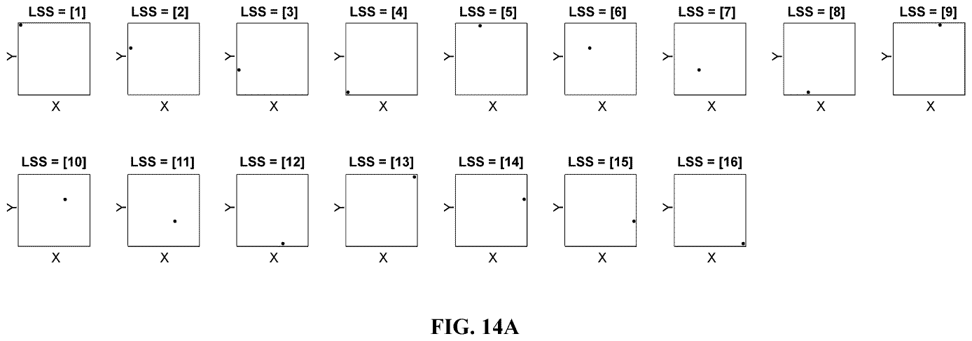

FIGS. 13A, 13B, and 13C illustrate exemplary properties of single-light-sharing-segment events, four selected two-light-sharing-segment events, and two selected three-light-sharing-segment events, in accordance with embodiment of the present disclosure.

In FIG. 13A, the property “probability” represents the fraction of incident 511 keV gamma rays that interact with the detector and deposit a total of 511 keV in a single light-sharing segment. Example LUT graphically displays a binary LUT for one particular single-light-sharing-segment event. As shown in the exemplary LUT, the gamma ray deposited all of its energy in light-sharing segment (LSS) #6. The corresponding positions of all of the single-light-sharing-segment peaks are shown as “+” in the LUT. Other properties in FIG. 13A will be discussed below with reference to FIGS. 13B and 13C.

FIG. 13B illustrates four selected two-light-sharing-segment events with the highest probabilities. In these instances, non-zero values in the LUTs appear as lines connecting two peaks corresponding to those light-sharing segments involved in the event. The property “maximum polygon edge length”, in this case, represents the distance between these two peaks. For two-light-sharing-segment events, the probability decreases as the distance between the two peaks corresponding to the involved light-sharing segments increases.

FIG. 13C illustrates two selected three-light-sharing-segment events with the highest probabilities. In these instances, non-zero values in the LUTs appear as triangular regions whose vertices are three peaks corresponding to those light-sharing segments involved in the event. The property “maximum polygon edge length”, in this case, represents the largest distance between two of the three peaks. The property “polygon area” represents the area of the triangular region. For cases where the polygon area is zero, these LUTs overlap with those in the two-light-sharing-segment events. For example, the LUT for LSS=[6 10 14] shown in FIG. 13C overlaps with the LUT for LSS=[6 14] shown in FIG. 13B. Given that the events that involve three light-sharing segments arranged consecutively in a line (“maximum polygon edge length”=2; “polygon area”=0), such as LSS [6 10 14], represent a very small fraction (<0.003) of the overall events depositing 511 keV, they can be safely neglected without practical consequence and thus omitted from the discussion of the disclosure.

Finally, the property “probability rank” represents the relative probability ranking of a specific type of events among all events. A probability rank of 1 represents the type of events with the highest probability, with increasing values indicating progressively less likely events.

Since each of the m first-level electronic units are connected to a single SiPM within each of the n light-sharing segments, if the values of Im,n are known, the values of the signals at each of the SiPMs in each of the segments in which energy was deposited during an event can be estimated. Let Eₘ represents the total signal level measured for the event in the m-th first-level electronic unit, n represents the number (ID) of a light-sharing segment determined as being involved in the event, and E_{m,n} denote the signal level at the SiPM within the n-th light-sharing segment that is connected to the m-th first-level electronic unit, then,

$${E}_{m , n} = {r}_{m , n} {E}_{m} \qquad ( 5 )$$

where m=I, II, III, and IV, and n takes on one to three different integer values in the range of [1, 16]. The remaining SiPMs, i.e., those within the light-sharing segments that are determined as not being involved in the event, are assumed to have zero signal.

Therefore, determining r_{m,n} is a key objective. In accordance with embodiments of the disclosure, r_{m,n} can be determined using the approach described below, beginning with identifying the light-sharing segment(s) within which the gamma-ray energy was deposited during the event.

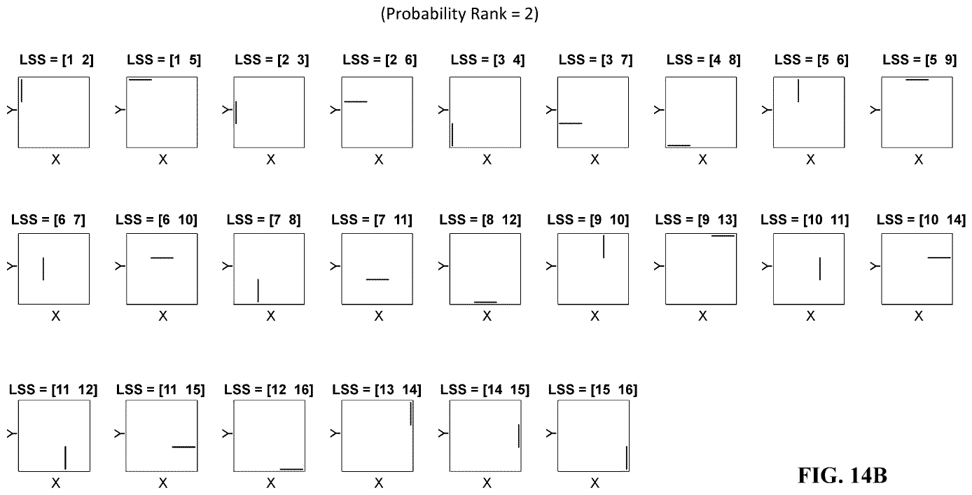

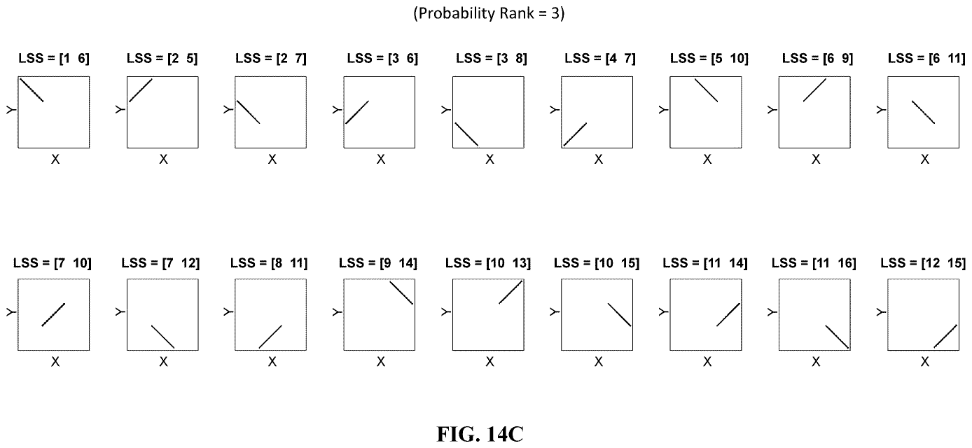

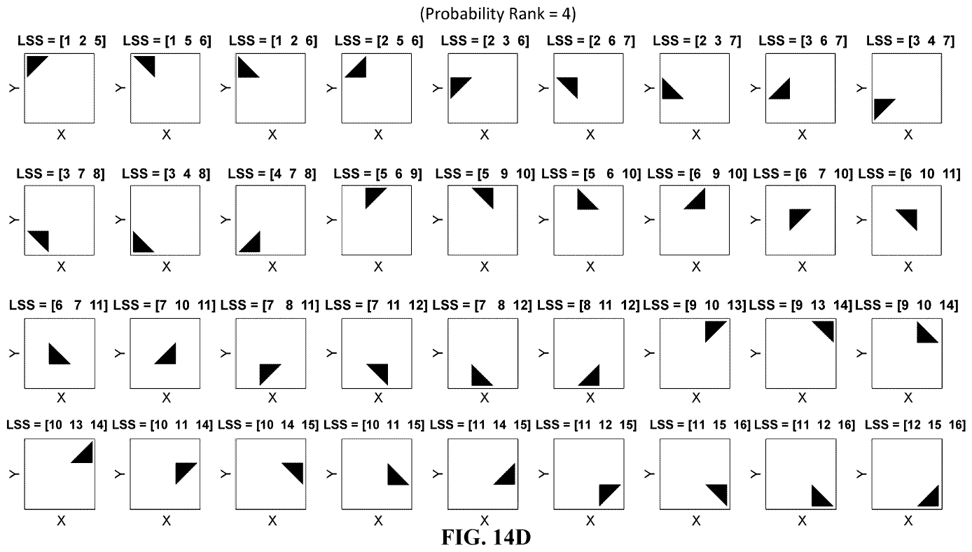

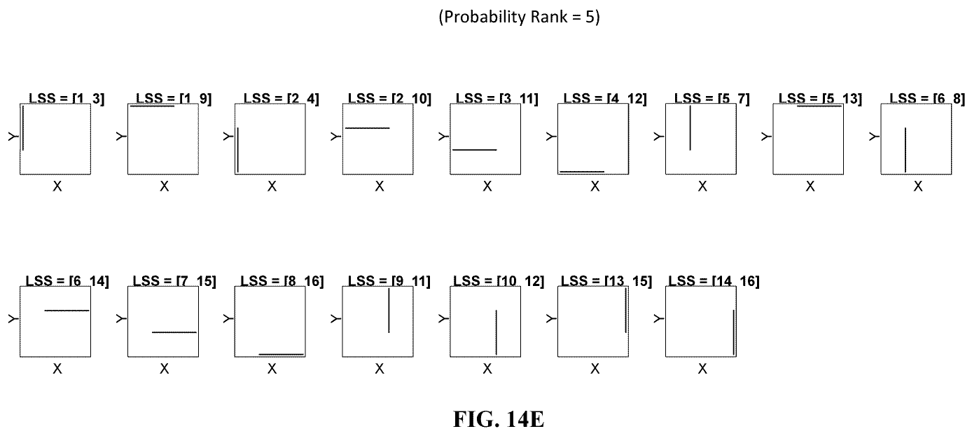

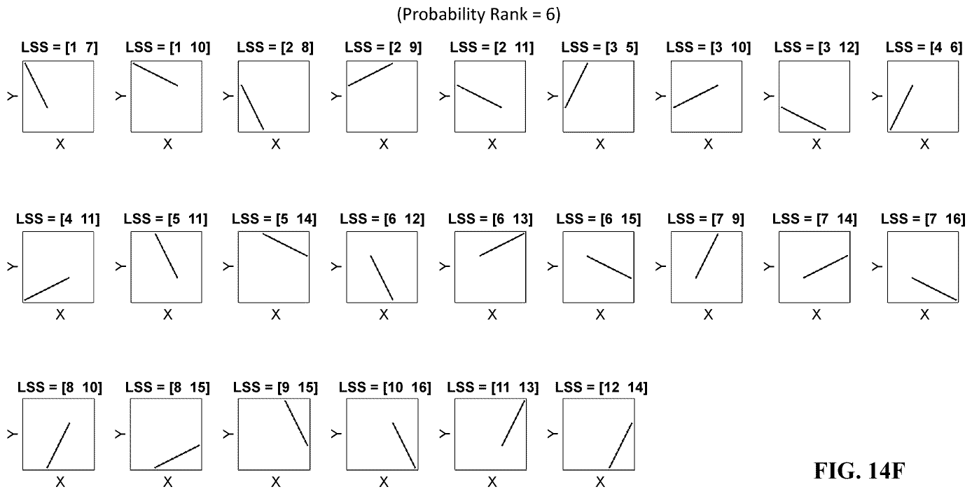

As mentioned earlier, during a calibration process, LUTs such as the ones shown in FIGS. 14A-14F are generated based on data collected for a great number of gamma-ray events. Although FIGS. 14A-14F only illustrate LUTs with probability ranks up to 6, those with higher-order probability ranks can be included as well. Also, as noted above, the four different first-level electronic units can have four different flood histograms, resulting in four different sets of LUTs. For the sake of simplicity and convenience, it is assumed in the following discussion that the flood histograms and, therefore, the LUTs are the same for all of the first-level electronic units.

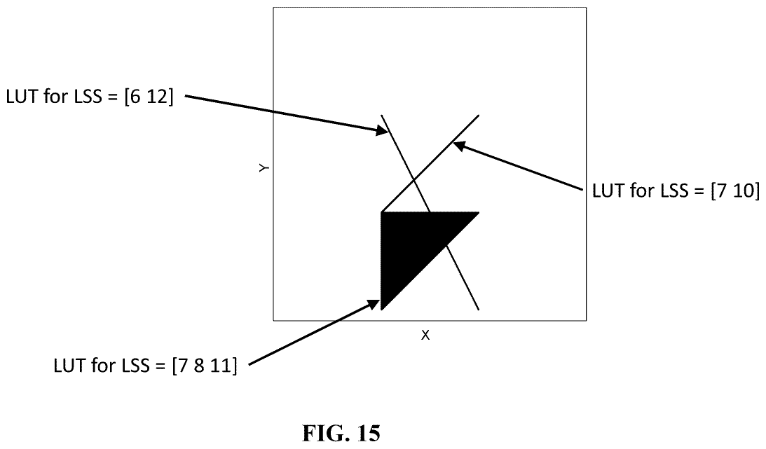

From the LUTs shown in FIGS. 14A-14F, it can be observed that many regions exhibit overlapping non-zero values from different LUTs. FIG. 15 shows an example where three LUTs for LSS=[6 12], [7 10], and [7 8 11] overlap to varying degrees. Since indexing into the LUTs with a single position pair (Xₘ′, Yₘ′) might result in non-zero values in multiple LUTs, there can be ambiguity in assigning an event to a single combination of light-sharing segments based solely on the (Xₘ′, Yₘ′) position pair.

According to embodiments of the present disclosure, a vote casting mechanism can be used to assign an event to a specific combination of one, two, or three light-sharing segments. Examples will be described below to illustrate this voting process.

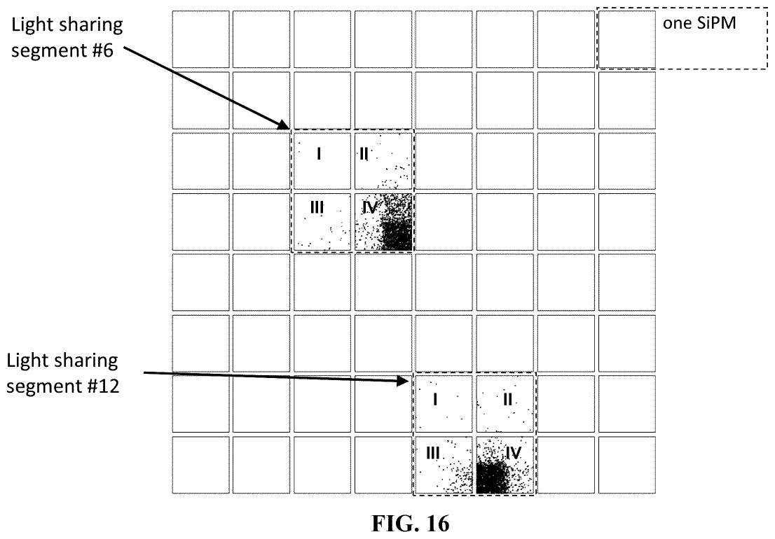

FIG. 16 shows an exemplary event obtained from a simulated data set. During this event, the 511 keV energy was split between two light-sharing segments, #6 and #12, through a combination of Compton scatter and photoelectric absorption interactions. The black dots in FIG. 16 represent the positions of detected optical photons relative to the individual SiPMs within boundaries of the light-sharing segments #6 and #12.

In this exemplary event, 255 keV was deposited in the light-sharing segment #6, and 256 keV in #12, indicating an almost equal energy split between the two light-sharing segments. However, the signal level split for each one of the four first-level electronic units I, II, III, and IV depends not only on the energy split, but also highly on the 3-D location of the interactions within the individual light-sharing segments.

For example, considering the first-level electronic unit IV, the numbers of photons on the corresponding SiPMs in the two light-sharing segments #6 and #12 were roughly equal, and thus the position of (X_{IV}′, Y_{IV}′) can be expected to lie roughly halfway between the peak positions corresponding to single-segment events in the light-sharing segments #6 and #12. However, considering the first-level electronic unit III, the corresponding SiPM in the light-sharing segment #12 received more photons compared to the SiPM in the light-sharing segment #6, and thus the position of (Xₘ′, Yₘ′) can be expected to appear closer to the peak corresponding to the single-segment events in the light-sharing segment #12. Similar discrepancies also occur for the first-level electronic units I and II. As a result, the measured positions with respect to the four first-level electronic units are likely to sample different points within the (X′, Y′) space and, therefore, sample different points in the LUTs.

Through the voting mechanism provided in this disclosure, each first-level electronic units can cast their votes for different combinations of light-sharing segments based on their respective values of (Xₘ′, Yₘ′). Specifically, each first-level electronic unit can cast multiple votes, with each vote for a combination that has a non-zero LUT value at the corresponding position pair (Xₘ′, Yₘ′).

The impact of noise in measurements must be considered in the implementation of this vote casting mechanism. For well-designed electronics, the electronic noise is generally sufficiently low as to be almost insignificant. However, when the signal level, Eₘ, in a particular first-level electronic unit, m, is low, dark counts from the SiPMs can produce significant variation in the measured position pair (Xₘ′, Yₘ′). More specifically, for SiPMs that detect thousands of photons (which is typical for an SiPM receiving a moderate to high signal level), the dark counts will have little influence on the measured position; while for SiPMs that receive a low signal level, such as those connected to the first-level electronic unit I in FIG. 16, the impact of the dark counts on the measured position can be significant.

Moreover, in the voting process, each first-level electronic unit should cast a vote for the actual or correct light-sharing-segment combination among which the energy of the gamma ray was distributed. Since the non-zero regions of the LUTs overlap, it is likely that the first-level electronic units will also cast votes for incorrect light-sharing-segment combinations. However, the correct combination should be the one receiving the most votes, or at least among a group of combinations receiving the most votes. If each first-level electronic unit bases its votes on a single position (Xₘ′, Yₘ′) in the LUTs, variation in position that results from dark counts can often cause that the first-level electronic units fail to cast votes for the correct light-sharing-segment combination, particularly when the light-sharing-segment combination is a single segment (where the non-zero LUT values are a small point-like region) or a two-segment combination (where the non-zero LUT values are a thin line-like region).

To deal with the inherent uncertainty in the voting process, embodiments of the disclosure base the voting on a region whose size is inversely dependent on the signal level in the given first-level electronic unit, rather than the single point (Xₘ′, Yₘ′). As a result, a first-level electronic unit with a lower signal level can use a larger area to cast its votes.

In one exemplary implementation, a circular voting region centered on (Xₘ′, Yₘ′) is used. The diameter, Dm, of the circular voting region varies with the signal level Eₘ of the m-th first-level electrical unit according to:

$${D}_{m} = α / {E}_{m} \qquad ( 6 )$$

where α is a constant used to establish the relationship between the diameter Dₘ of the circular region and the measured signal level Eₘ in the corresponding first-level electronic unit. To ensure that each first-level electronic unit casts a vote for the actual light-sharing-segment combination among which the energy of the gamma ray was distributed, the scaling factor α is chosen so that the circular region intersects the non-zero LUT region for the actual correct light-sharing-segment combination. That is, instead of numerically representing the standard deviation or variance of the position due to dark counts (which would result in a significant fraction of the cases not casting a vote for the correct combination), a is chosen to be larger than these conventional measures. This ensures coverage of the vast majority (e.g., >99%) of correct actual non-zero LUT regions.

According to embodiments of the disclosure, a can be determined from simulated data. For example, a can be adjusted until the proportion of correctly identified light-sharing segments for a large number of events is maximized. In this approach, it is important to tune the dark count rate in the simulation to closely approximate the rate observed in measurements, as this dark count rate is the main contributor to errors in the voting process. Alternatively, α can be varied until an expected distribution of different probability ranks is achieved. Other approaches are also available; for instance, PET image reconstructions can be performed with the simulation data processed using different values of a. Then, the value of a that produces the best image quality can be chosen.

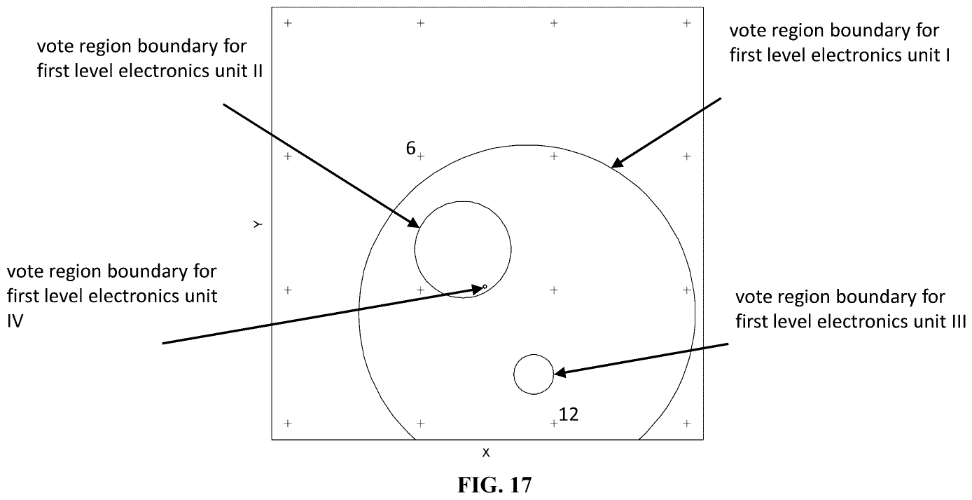

For the exemplary event shown in FIG. 16, FIG. 17 illustrates the corresponding boundaries of the voting regions for the four first-level electronic units I-IV. The locations of the single-segment peaks are indicated by “+” for reference. Each first-level electronic unit casts a vote for each LUT in which a non-zero LUT value occurs within the corresponding voting region for that first-level electronic unit.

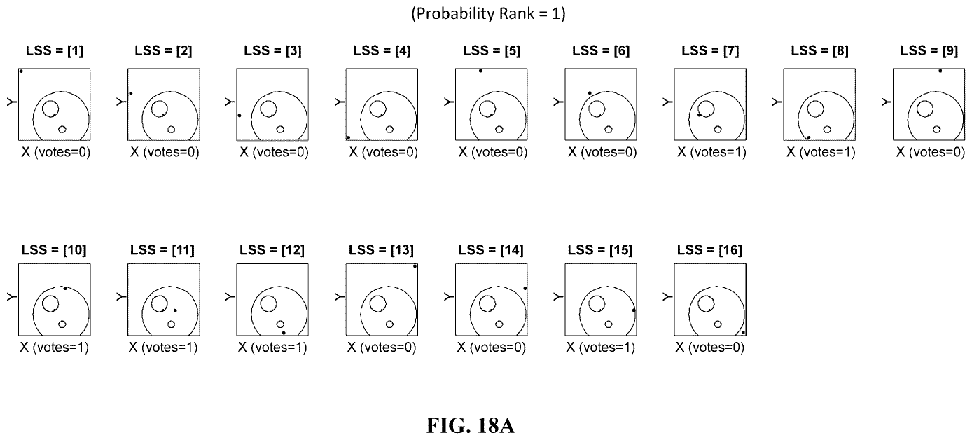

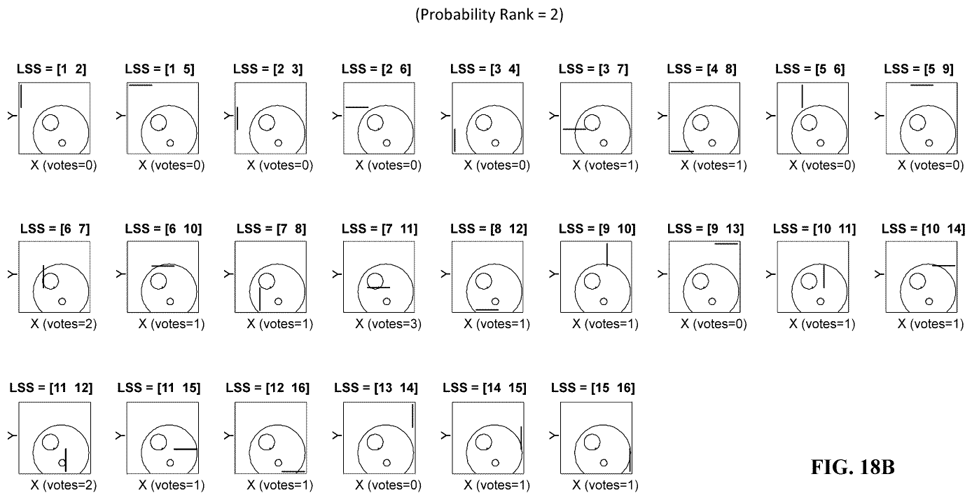

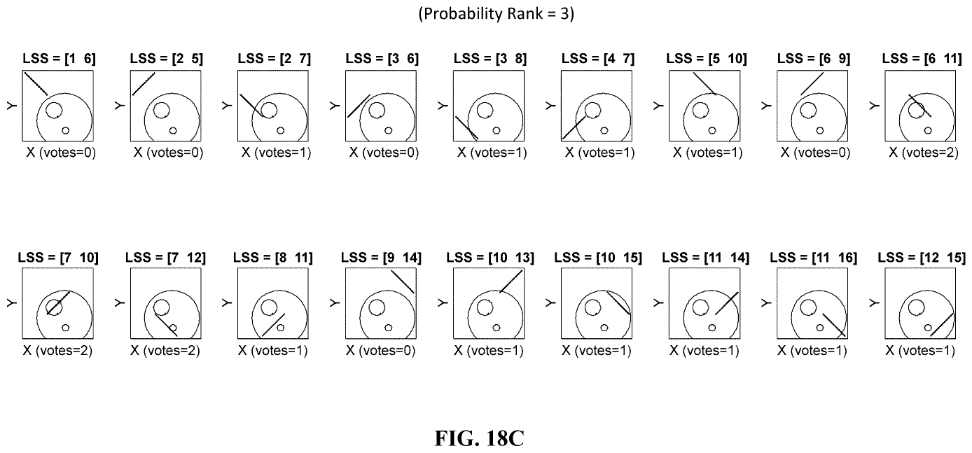

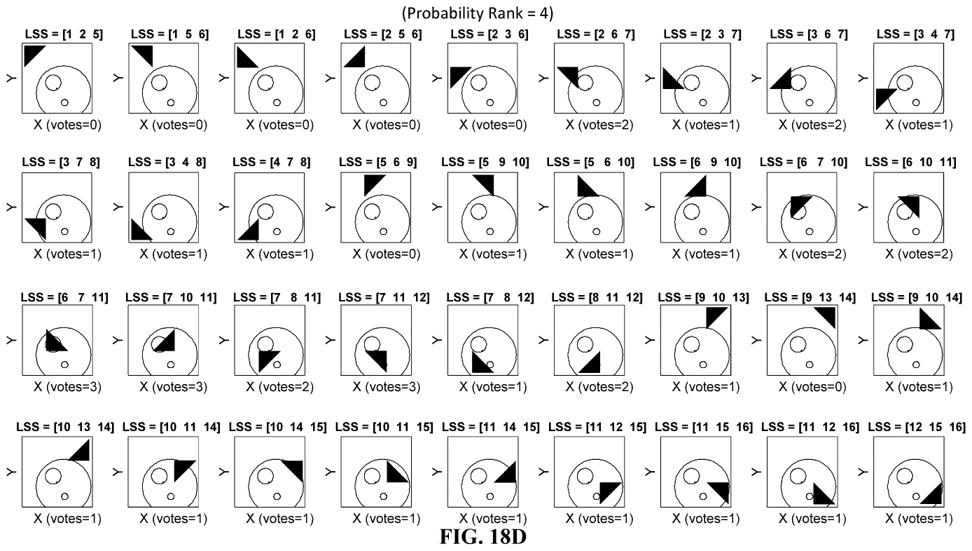

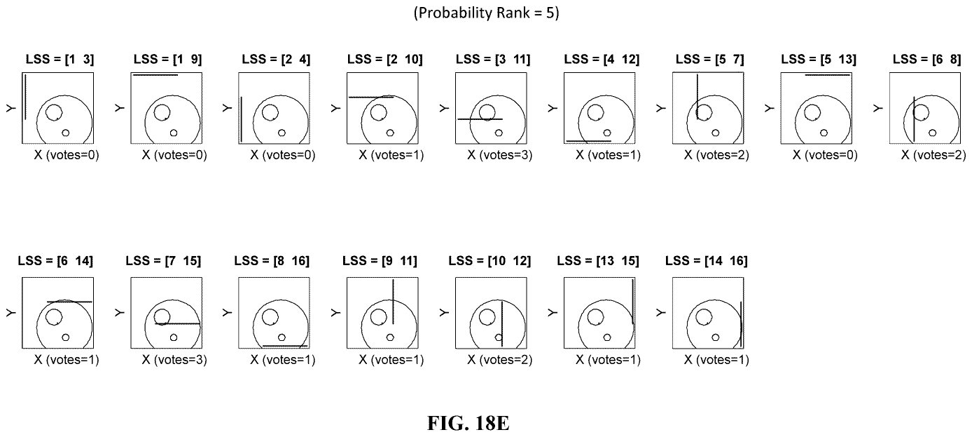

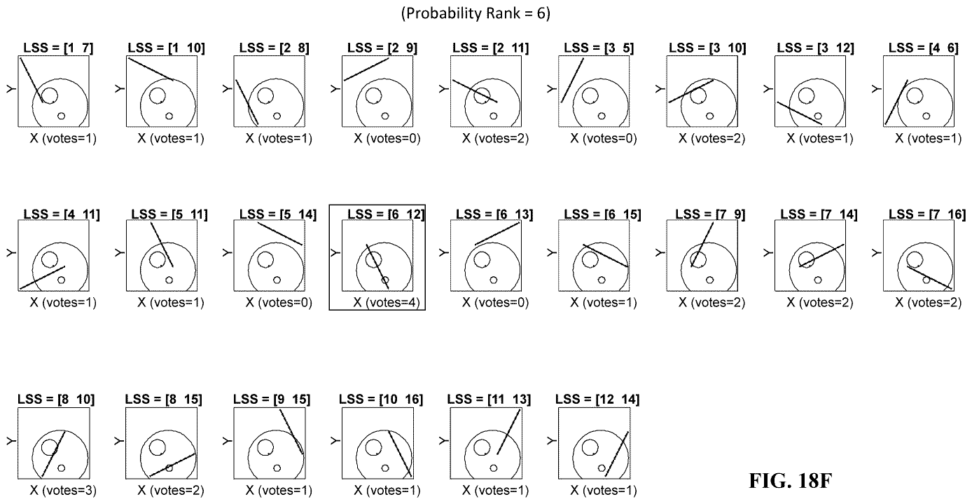

FIGS. 18A-F show the boundaries of the voting regions within the respective LUTs. The total votes for each LUT are shown on the x-axis labels. Among all LUTs, only one LUT shown in FIG. 18F, which corresponds to the LSS=[6 12] events, received 4 votes. Therefore, it can be determined from the measurements that the event distributed its energy between these two light-sharing segments, namely segments #6 and #12.

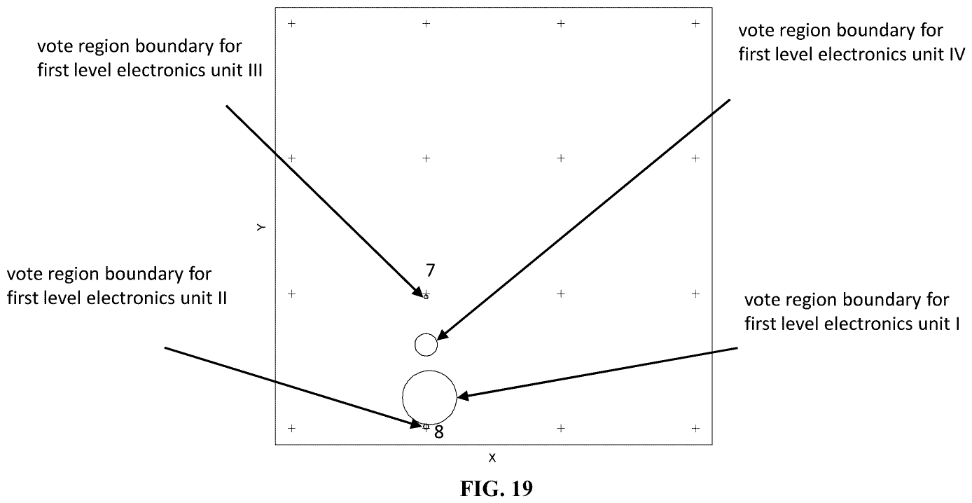

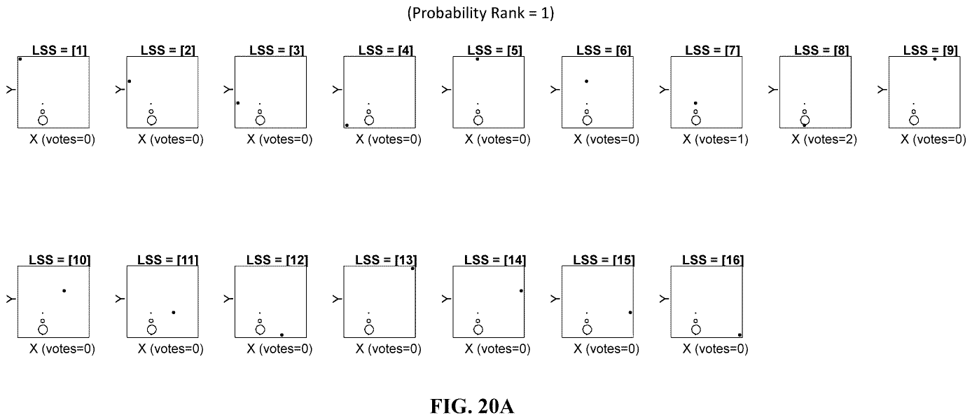

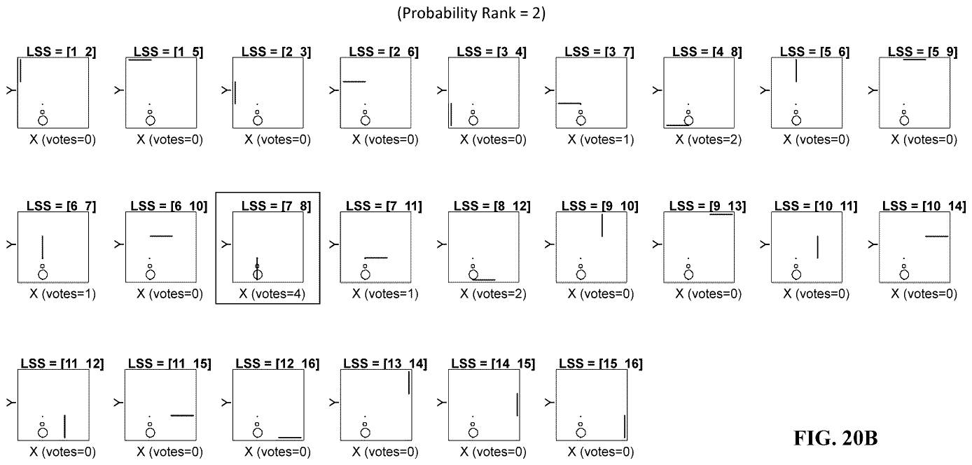

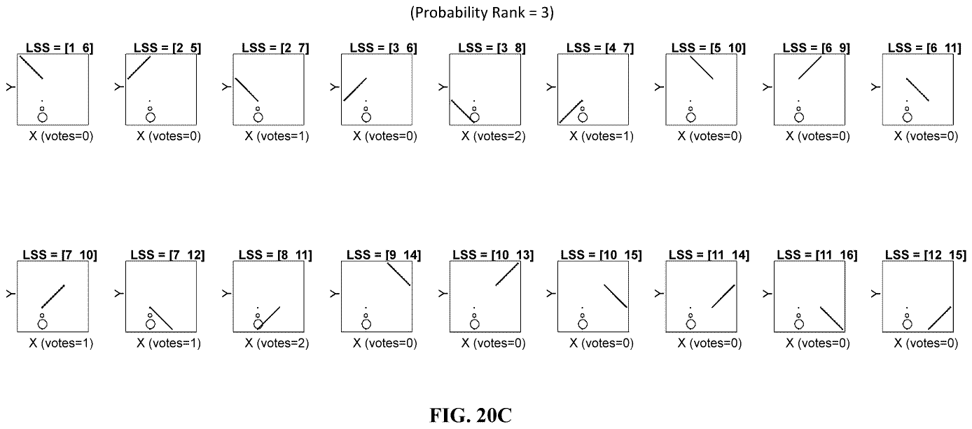

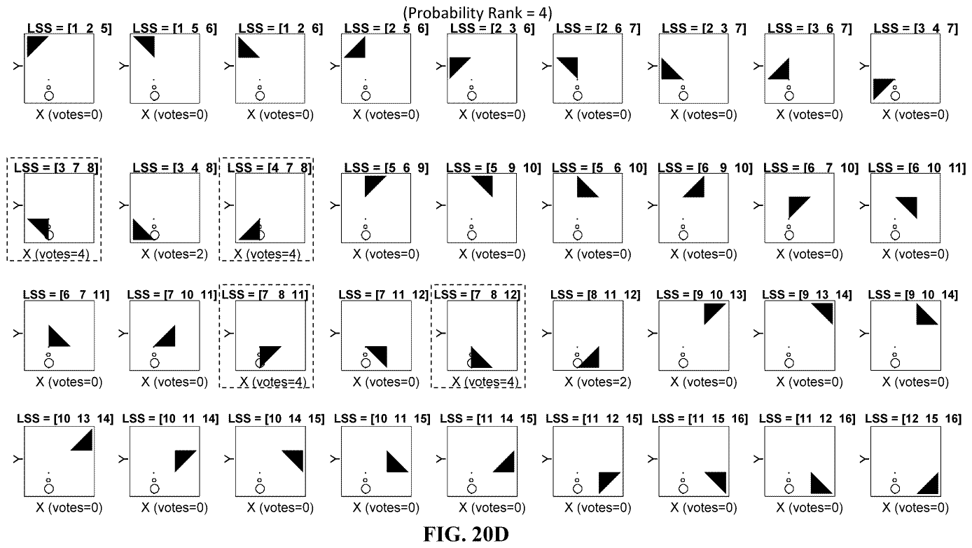

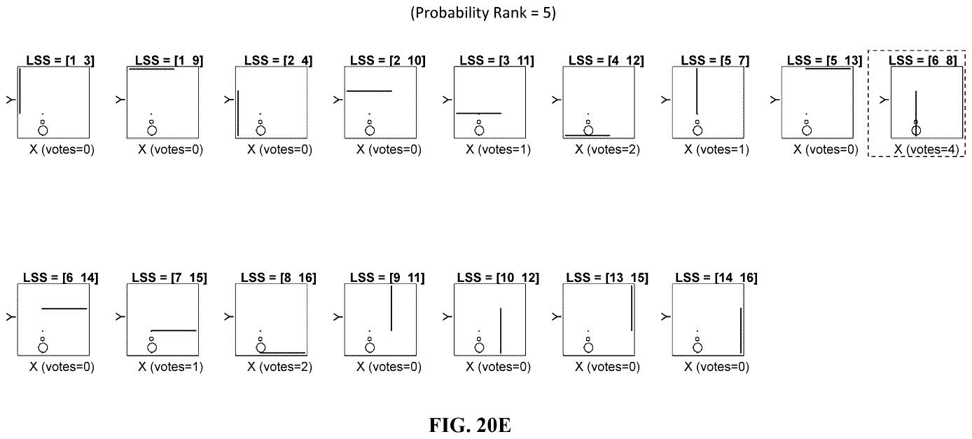

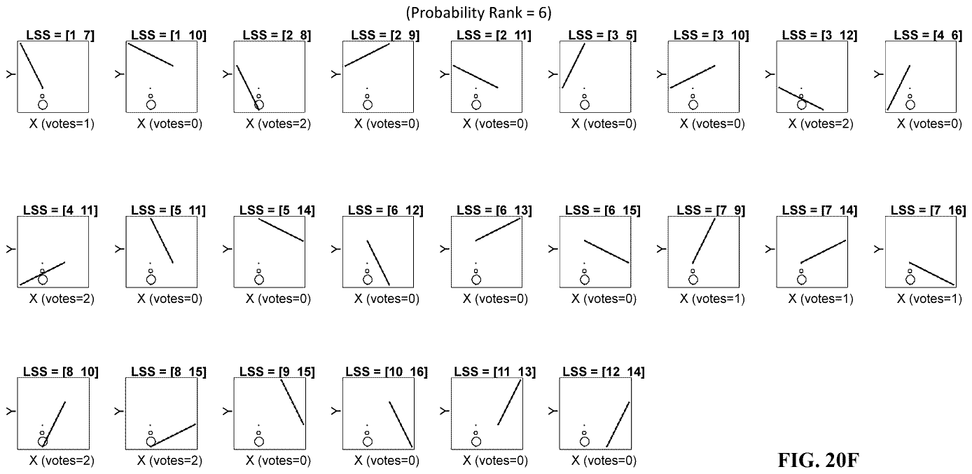

Consider an exemplary event where a gamma ray deposits all its 511 keV energy in the light-sharing segments #7 and #8 through a combination of Compton scatter and photoelectric absorption interactions. FIG. 19 shows the boundaries of the voting regions for each of the first-level electronic units. FIGS. 20A-F illustrate the voting region boundaries in the LUTs, with the number of votes cast for each LUT indicated on the x-axis labels.

In this scenario, six LUTs each receive four votes. As a tie occurs after all votes have been cast, the property, probability rank, of the LUTs can be used as a tie-breaker. Among the six LUTs, one has a probability rank of 5, and four have a probability rank of 3. Only one LUT, shown in FIG. 20B, corresponding to the LSS=[7 8] events, receives four votes and has a probability rank of 2. Since a lower probability rank indicates a more probable light-sharing segment combination, the event can be correctly assigned to LSS=[7 8].

The two examples describe above represent the majority of cases. In these scenarios, either a single LUT (corresponding to a single LSS combination) receives the most votes outright, or there is a tie in total number of votes, with only one LUT among those receiving the most votes having the lowest probability ranking, indicating the most probable light-sharing segment combination.

In very rare instances, there may be two LUTs that both receive the most votes and also have the same probability ranking. In such cases, additional measures are necessary to break the tie. These scenarios generally involve equal-probability-ranking events where non-zero LUT regions overlap in areas that do not contain single-light-sharing-segment peaks (LUTs with a probability rank=1).

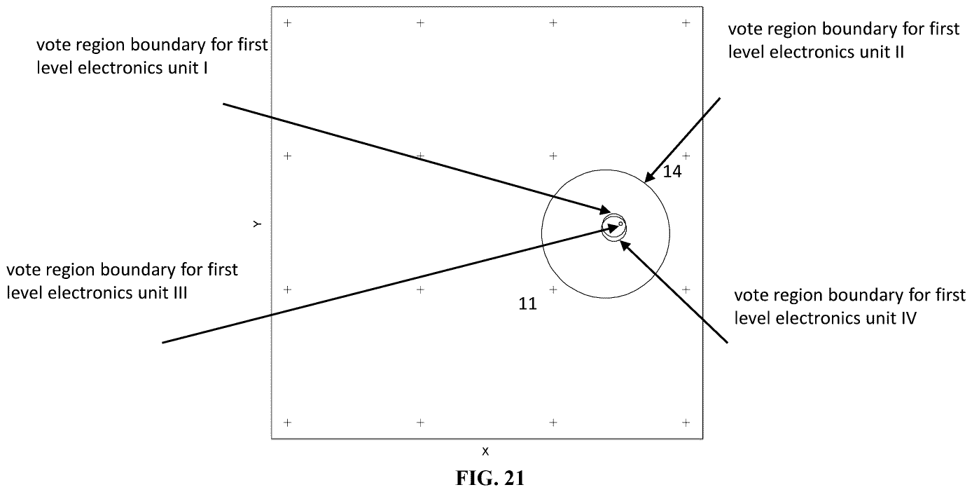

The first case where an additional tie breaking method is required involves overlapping “diagonal” two-light-sharing-segment LUTs. These can occur in LUTs with a probability rank=3 and LUTs with a probability rank=6, for example. FIG. 21 shows the corresponding boundaries of the voting regions for each of the first-level electronic units for a simulated case where this type of tie-breaker is needed. From the simulation, it is known that the event actually deposited energy in the light-sharing segments #11 and #14.

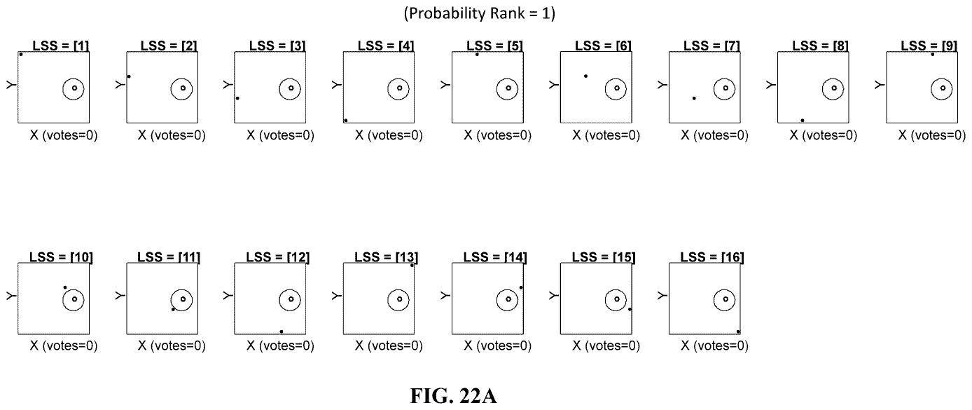

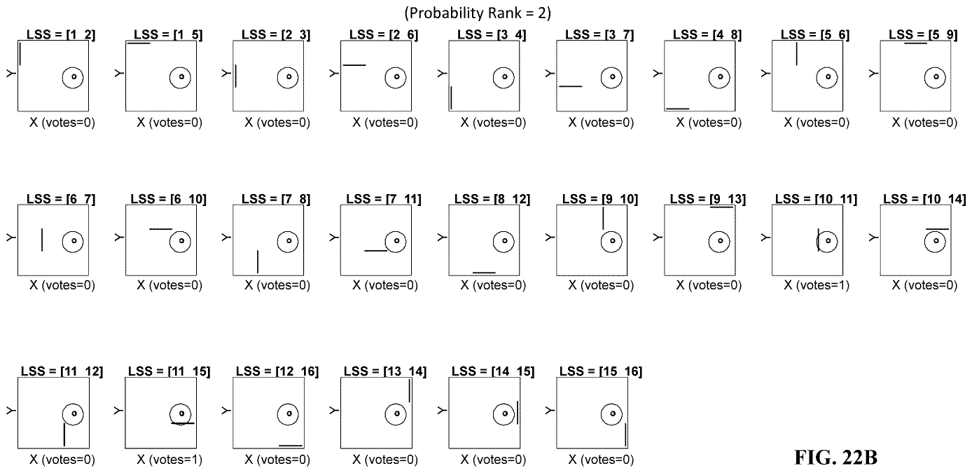

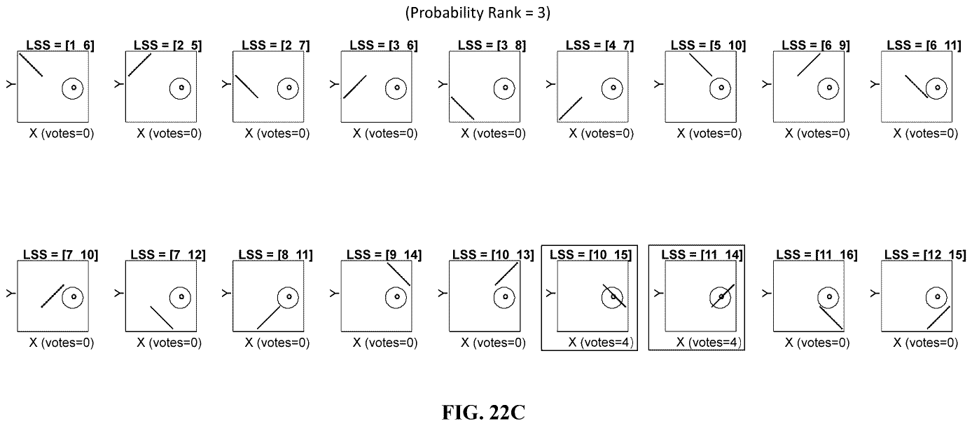

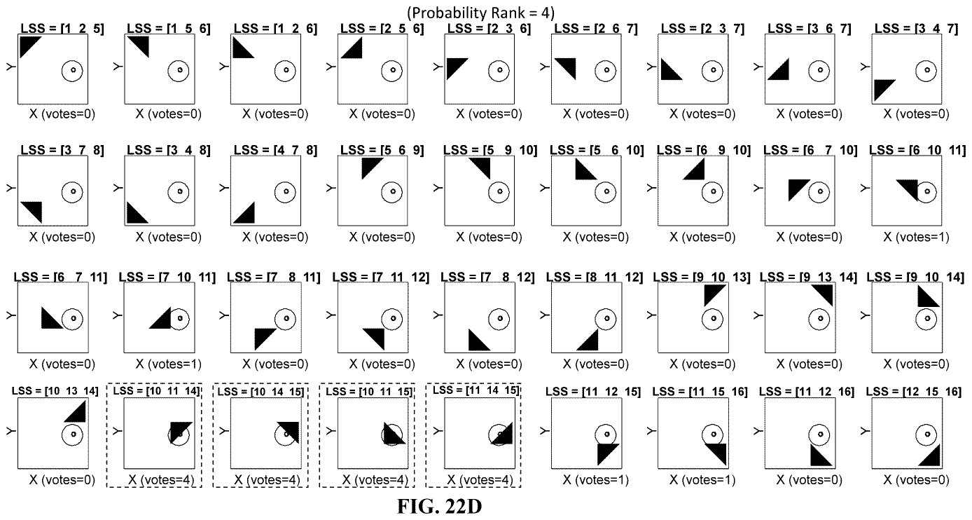

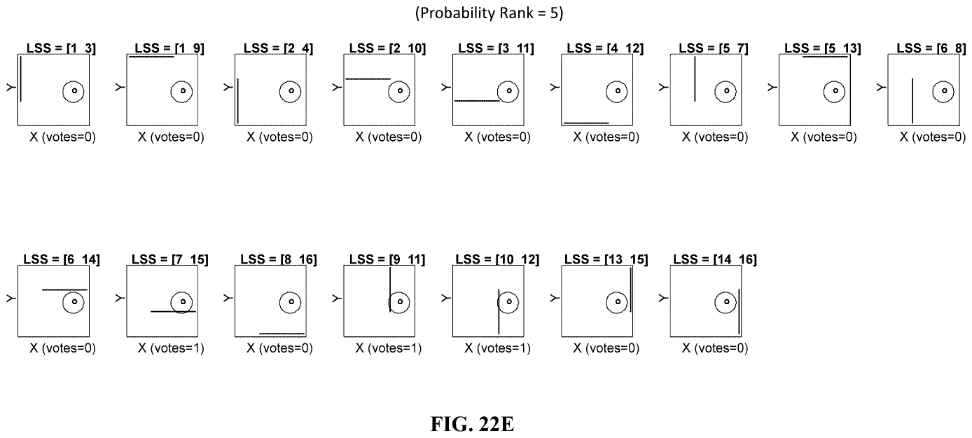

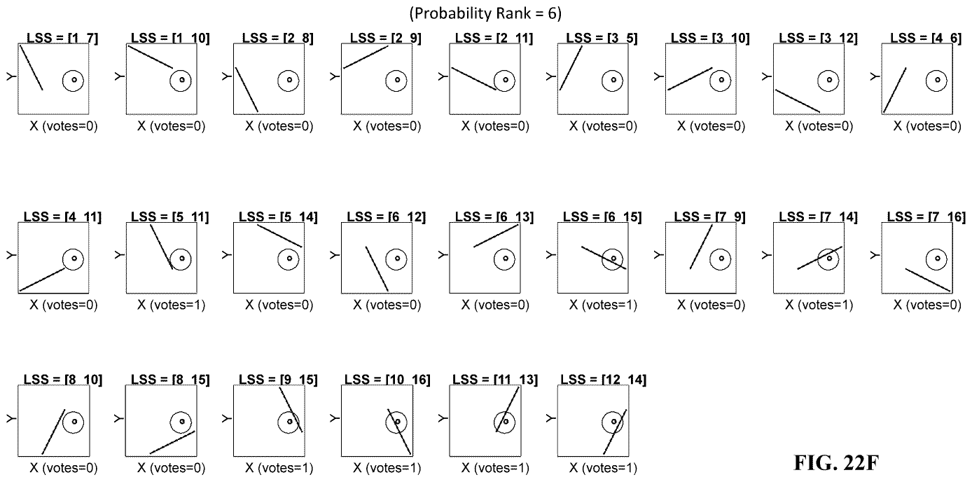

In FIGS. 22A-F, the boundaries of the voting regions are shown in the LUTs, and the number of votes cast for each LUT is shown on the x-axis labels. In this case, there are a total of six LUTs that each receive 4 votes, but the four LUTs with a probability rank=4 (shown in FIG. 22D) immediately can be eliminated because the two LUTs (LSS=[10 15] and LSS=[11 14]; shown in FIG. 22C) with a probability rank=3 are more likely to be the correct result, based on the difference in probability ranking. Since the remaining two LUTs have the same probability ranking, however, an additional tie-breaker is required.

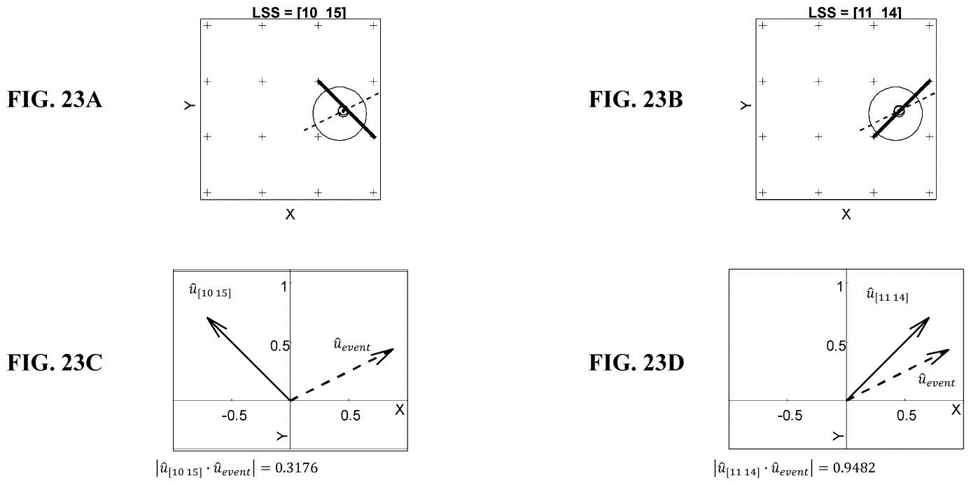

FIGS. 23A-23D illustrate a scenario using an additional tie-breaker for overlapping “diagonal” two-light-sharing-segment LUTs, in accordance with one embodiment of the disclosure. FIG. 23A shows the LUT for LSS=[10 15], along with the boundaries of the voting regions, and a linear fit indicated by a dashed line. FIG. 23B shows the LUT for LSS=[11 14], along with the boundaries of the voting regions, and the same linear fit as shown in FIG. 23A, which is indicated by a dashed line.

This additional tie-breaker considers the correlation between the direction of the non-zero LUT segments and a linear fit to the centers of the voting regions [i.e., (Xₘ′, Yₘ′)]. When performing the least-squares linear fit, the errors between the line and the points (Xₘ′, Yₘ′) can be weighted by 1/Dₘ².

In FIG. 23C, the unit vectors corresponding to the direction of the LSS=[10 15] LUT is shown as a solid vector and the unit vector corresponding to the direction of the linear fit is shown as a dashed vector. As a measure of the correlation between the directions, the absolute value of the dot product of the unit vectors can be calculated. For the LSS=[10 15] LUT and this event, the absolute value of the dot product is 0.3176.

FIG. 23D shows the unit vector corresponding to the direction of the LSS=[11 14] LUT as a solid vector and the unit vector corresponding to the direction of the linear fit is shown as a dashed vector. For the LSS=[11 14] LUT and this event, the absolute value of the dot product is 0.9482. The larger absolute value of the dot product for the latter case indicates a better match between the event and the LUT for LSS=[11 14]. Therefore, the event can be correctly assigned to LSS=[11 14].

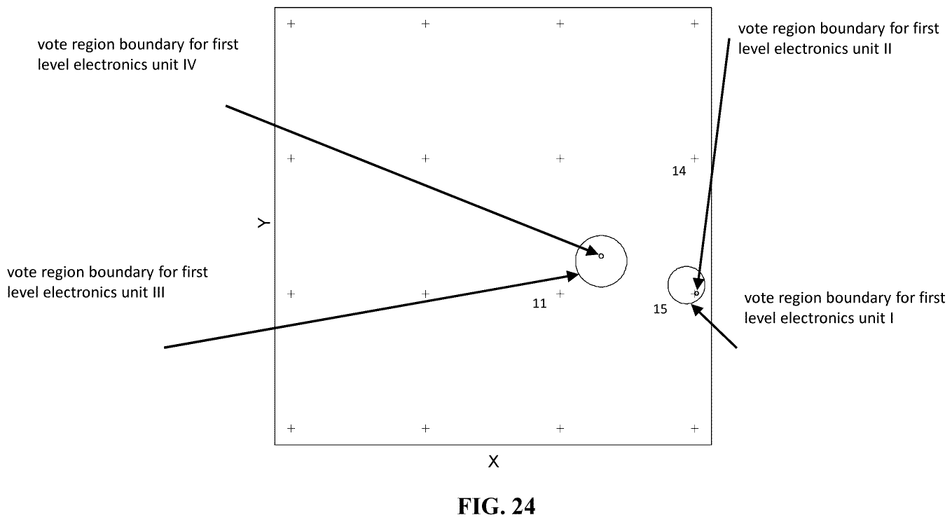

The second case where an additional tie breaking method is required involves overlapping “triangular” three-light-sharing-segment LUTs. These can occur in LUTs with a probability rank=4, for example. FIG. 24 shows the corresponding boundaries of the voting regions for each of the first-level electronic units for a simulated case where this second type of tie-breaker is needed. From the simulation, it is known that the event actually deposited energy in the light-sharing segments #11, #14, and #15.

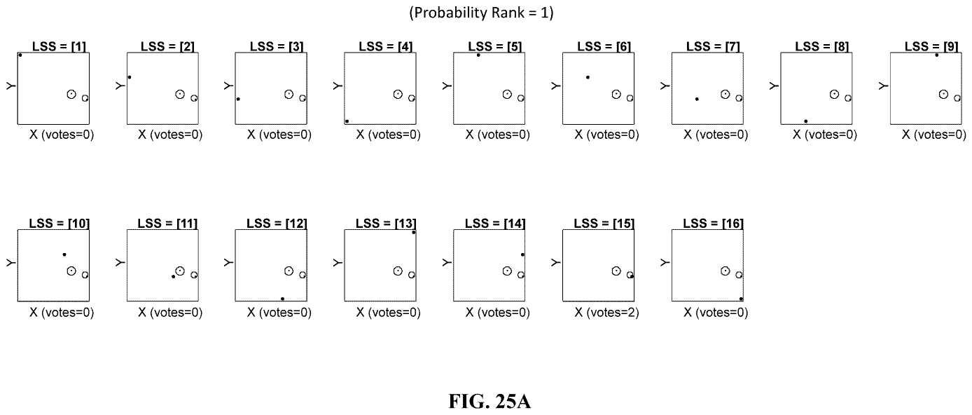

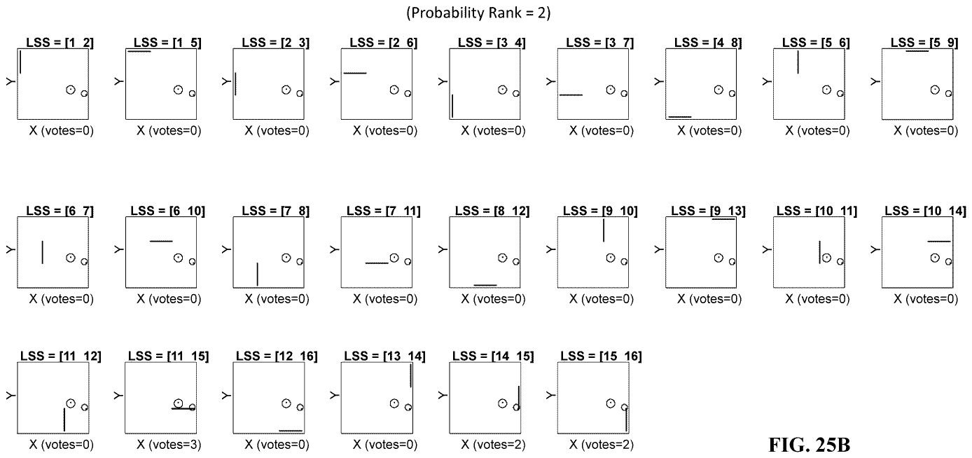

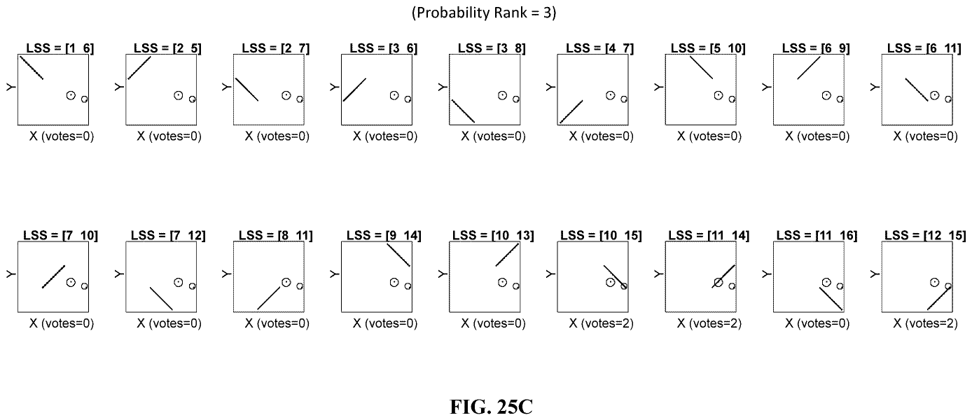

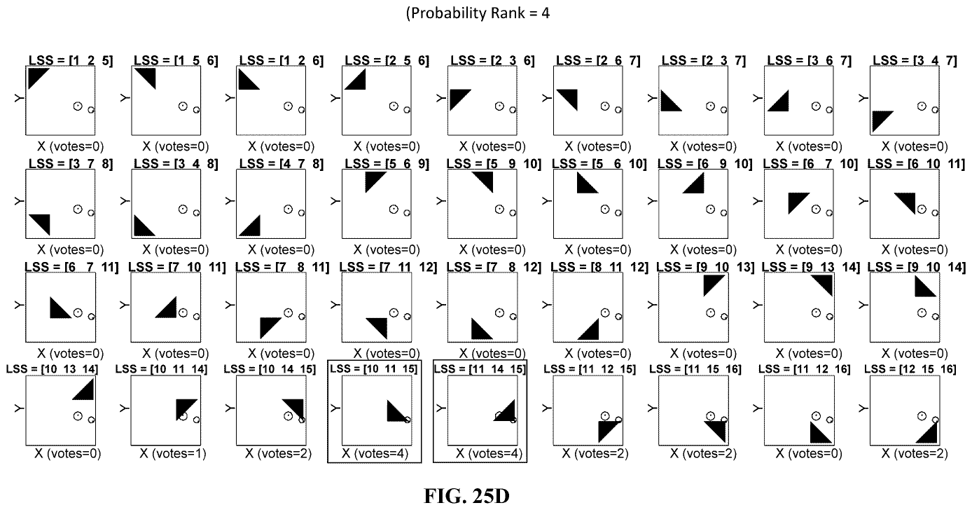

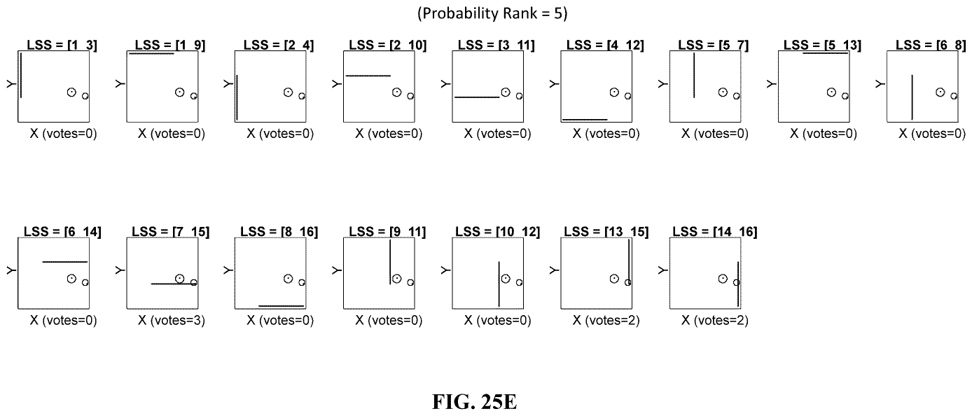

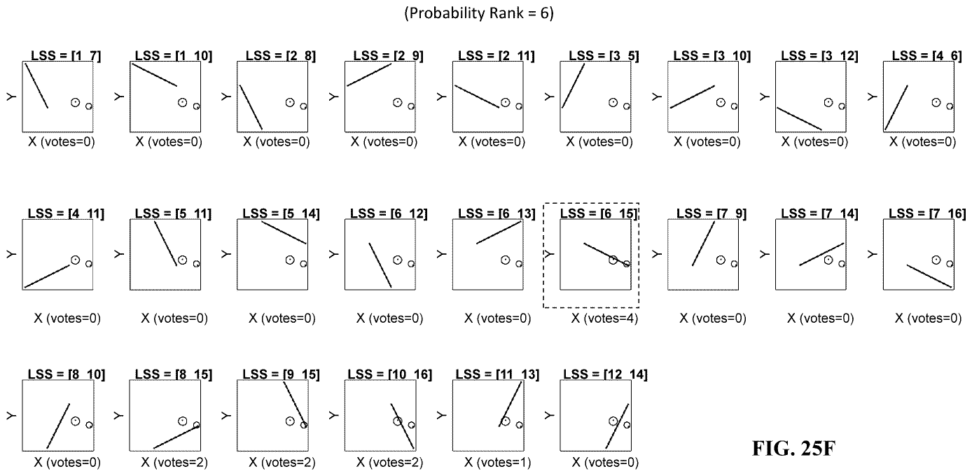

In FIGS. 25A-F, the boundaries of the voting regions are shown in the LUTs, and the number of votes cast for each LUT is shown on the x-axis labels. In this case, there are a total of three LUTs that each receive 4 votes. As in the previous cases, the one LUT with a probability rank=6 (shown in FIG. 25F) immediately can be eliminated, because the two LUTs (LSS=[10 11 15] and LSS=[11 14 15]; shown in FIG. 25D [NOTE: FIG. 25D appears to be missing from the PPT file containing the figures.]) with a probability rank=4 are more likely to be the correct result, based on the difference in probability ranking. Since the remaining two LUTs have the same probability rank, however, an additional tie-breaker is required.

The tie-breaker for the overlapping three-light-sharing-segment LUTs is based on the observation that in many three-light-sharing-segment events, the amount of energy deposited in one of the light-sharing segments is relatively low. For example, when considering only events that deposit a total of 511 keV in three light-sharing segments, in approximately 60% of the cases, one of the light-sharing segments receives energy less than 65 keV. Combined with the variation in light sharing within the segment, the result is that most three-light-sharing-segment events have a signal-level-weighted position (Xₘ′, Yₘ′) that lies near a two-segment-event non-zero LUT region on the boundary of the corresponding three-segment event. This is true because when an SiPM in one light-sharing segment has a low signal level, the signal-level-weighted position (Xₘ′, Yₘ′) does not differ much from the position that would be calculated using only the two highest signal levels for that first-level electronic unit. Since a voting region (rather than a point) is used to account for uncertainty in the measured position due to noise, it becomes highly likely for (Xₘ′, Yₘ′) near a two-segment-event non-zero LUT region to cast a vote for that LUT.

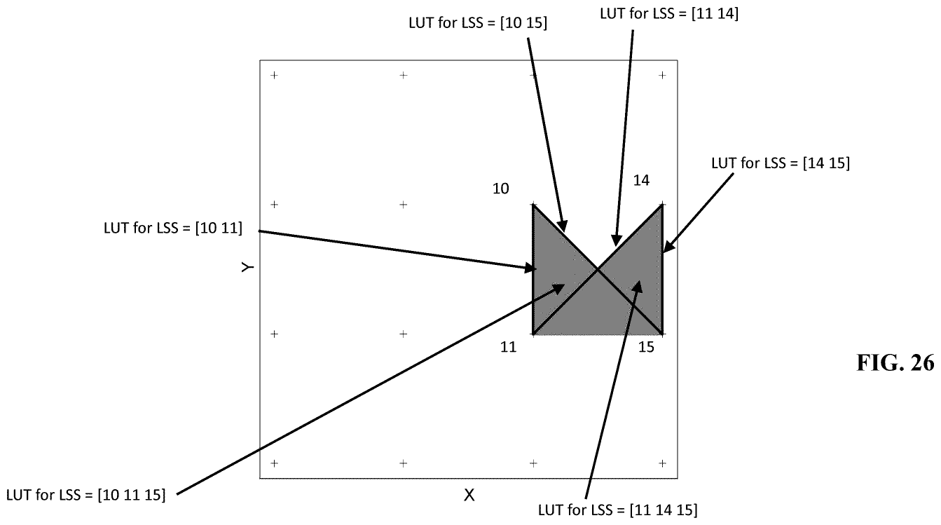

In order to differentiate between two overlapping three-light-sharing-segment LUTs, as a tie-breaker, only votes cast for non-common two-segment events on the boundary of the three-segment events that have tied need to be considered. Two common light-sharing segments that are shared by the two overlapping three segment event are first identified. For the example shown in FIG. 26, LSS=[10 11 15] and LSS=[11 14 15] have the light-sharing segments #11 and #15 in common. Then, the non-common two segment events on the border of the non-zero LUT region of LSS=[10 11 15] are determined, namely LSS=[10 11] and LSS=[10 15]. Similarly, for LSS=[11 14 15], the non-common two segment events on the border of the non-zero LUT region are determined as LSS=[11 14] and LSS=[14 15]. In FIG. 26, the two overlapping three-segment LUTs are indicated in gray, and the four non-common two-segment LUTs are shown in black.

The votes from any first-level electronic units that cast votes for the single-light-sharing-segment events of LSS #11 or #15 can be eliminated, because these votes will provide no differentiating information. The reason is that they will also cast votes for one of the boundary two-segment events corresponding to each of the three segment events in question. Referring to FIGS. 24 and 25A, the votes for m=I and m=II are eliminated, because they both cast votes for LSS=[15] and would therefore cast non-differentiating votes in the tie-breaker voting.

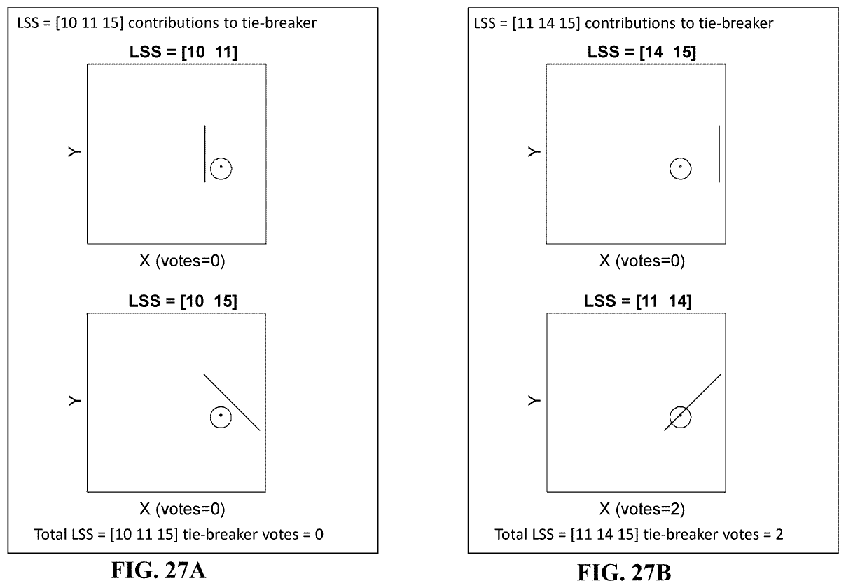

FIGS. 27A and 27B illustrate the result of the overlapping three-segment tie-breaker voting. In FIG. 27A, the non-common two-segment events on the border of the non-zero region of the LUT for LSS=[10 11 15] have their votes summed for LSS=[10 11 15]. In FIG. 27B, the non-common two-segment events on the border of the non-zero region of the LUT for LSS=[11 14 15] have their votes summed for LSS=[11 14 15]. Because LSS=[11 14 15] received more votes in the tie-breaker voting, the event can be correctly assigned to LSS=[11 14 15].

Using simulation data that includes reasonable levels of dark counts from the SiPMs, the vote casting mechanism described above can achieve over 98% accuracy in determining the correct combination of light-sharing segments. Notably, the accuracy can be further improved by including even less likely occurring events, such as by adding more LUTs with higher probability ranking.

An alternative approach that can yield more accurate results than circular voting regions is to use elliptical voting regions, where the semi-major axis length, semi-minor axis length, and angle of the semi-major axis depend on (Xₘ′, Yₘ′). This approach provides better results because dark counts tend to pull low-signal-level interactions in corner LSSs toward the center of the flood histogram, making the uncertainty region more elliptical. In contrast, for low-signal-level interactions in the central LSSs, the effect of dark counts is more isotropic, resulting in more circular uncertainty regions.

In some embodiments of the disclosure, in order to improve the computational speed of the process, votes from first-level electronic units with signal levels below a certain threshold can be eliminated. This would not affect the voting process significantly, as these units cast many votes and therefore provide minimal additional information.

In some embodiments, a fifth vote based on the energy-weighted average position of the m measured positions can be included in the voting process. This fifth vote would have the lowest uncertainty, as it is generated from all of the available signals. However, while this additional vote can improve performance, it comes at the cost of extra computation.

Instead of relying on LUTs, the identification of the involved light-sharing segment(s) can be achieved through analytical calculation methods. These methods can be based on determining distances from and overlaps with polygons, including degenerate polygons with one or two vertices.

Once the light-sharing segment or segments in which energy was deposited have been determined, the next step is to determine the r_{m,n} values, which represent the fraction of the total signal reaching the n-th light-sharing segment in the m-th first-level electronic unit, where m=I, II, III, and IV, and n takes on one to three different integer values in the range of [1, 16].

These r_{m,n} values can be determined by solving a set of linear equations in a least-squares sense for the m first-level electronic units. The set of linear equations is based on equations (2)-(4) above and can be written as:

$$Ar = B \qquad ( 7 )$$

where r is a vector to be solved for, and A and B are matrices described below.

Let nLSS1, nLSS2, and nLSS3 denote the one, two, or three light-sharing segments involved in the event, which have been identified using the voting process described above. For the scenario where only one light-sharing segment is involved, r_{m,nLSS1} is to be solved for from A and B obtained from calibrations and measurements, respectively. The equations for the m first-level electronic units are as follows:

$$A = [ \begin{matrix}{xpeak}_{m , nLSS 1} \\ {ypeak}_{m , nLSS 1} \\ 1\end{matrix} ] \qquad ( 8 )$$

$$B = [ \begin{matrix}{X}_{m}^{′} \\ {Y}_{m}^{′} \\ 1\end{matrix} ] \qquad ( 9 )$$

$$and$$

$$r = [ {r}_{m , nLSS 1} ] \qquad ( 10 )$$

For the case of one light-sharing segment, the solution is r_{m,nLSS1}=1. Then, the signal levels E_{m,n} at the SiPMs included in the one light-sharing segment can be determined from the measured signal levels Eₘ using Equation (5). The signal levels at all other SiPMs in the SiPM array are assumed to be zero.

For the case of two light-sharing segments, r_{m,nLSS1} and r_{m,nLSS2} are to be solved for from A and B obtained from calibrations and measurements, respectively. The equations for the m first-level electronic units are as follows:

$$A = [ \begin{matrix}{xpeak}_{m , nLSS 1} & {xpeak}_{m , nLSS 2} \\ {ypeak}_{m , nLSS 1} & {ypeak}_{m , nLSS 2} \\ 1 & 1\end{matrix} ] \qquad ( 11 )$$

$$B = [ \begin{matrix}{X}_{m}^{′} \\ {Y}_{m}^{′} \\ 1\end{matrix} ] \qquad ( 12 )$$

$$and$$

$$r = [ \begin{matrix}{r}_{m , nLSS 1} \\ {r}_{m , nLSS 2}\end{matrix} ] \qquad ( 13 )$$

Standard techniques can be used to solve this set of linear equations in a least squares sense, with a constraint that all r_{m,n} are in the range [0, 1]. Again, the signal levels E_{m,n} at the SiPMs included in the two light-sharing segments can be determined from the measured signal levels Eₘ using Equation (5). The signal levels at all other SiPMs in the SiPM array are assumed to be zero.

Finally, for the case of three light-sharing segments, r_{m,nLSS1}, r_{m,nLSS2}, and r_{m,nLSS3} are to be solved for from A and B obtained from calibrations and measurements, respectively. The equations for the m first-level electronic units are as follows:

$$A = [ \begin{matrix}{xpeak}_{m , nLSS 1} & {xpeak}_{m , nLSS 2} & {xpeak}_{m , nLSS 3} \\ {ypeak}_{m , nLSS 1} & {ypeak}_{m , nLSS 2} & {ypeak}_{m , nLSS 3} \\ 1 & 1 & 1\end{matrix} ] \qquad ( 14 )$$

$$B = [ \begin{matrix}{X}_{m}^{′} \\ {Y}_{m}^{′} \\ 1\end{matrix} ] \qquad ( 15 )$$

$$and$$

$$r = [ \begin{matrix}{r}_{m , nLSS 1} \\ {r}_{m , nLSS 2} \\ {r}_{m , nLSS 3}\end{matrix} ] \qquad ( 16 )$$

Once again, standard techniques can be used to solve this set of linear equations in a least squares sense, with a constraint that all r_{m,n} are in the range [0, 1]. Then, the signal levels E_{m,n} at the SiPMs included in the three light-sharing segments can be determined from the measured signal levels Eₘ using Equation (5). The signal levels at all other SiPMs in the SiPM array are assumed to be zero.

After determining the individual signal levels at SiPMs of the SiPM array, energy, position (in 3-D; including a DOI index), and timing signals of the event can be determined using various methods. For instance, considering a scenario where the event deposited all its energy within two light-sharing segments, e.g., #2 and #6. In this example, Anger logic processing can be applied to the obtained signals E_{I,2}, E_{I,6}, T_{I} (from the first-level electronic unit I), E_{II,2}, E_{II,6}, T_{II} (from the first-level electronic unit II), E_{III,2}, E_{III,6}, T_{III} (from the first-level electronic unit III), E_{IV,2}, E_{IV,6}, and T_{IV} (from the first-level electronic unit IV).

For the interaction that happened within the light-sharing segment #2 during the event, the energy and 3D position signals can be calculated as follows:

$${E}_{2} = the total energy signal in light - sharing segment \#2 = {E}_{I , 2} + {E}_{II , 2} + {E}_{III , 2} + {E}_{IV , 2} \qquad ( 17 )$$

$${X}_{2} = the X position within the light - sharing segment \#2 = [ ( {E}_{II , 2} + {E}_{IV , 2} ) - ( {E}_{I , 2} + {E}_{III , 2} ) ] / {E}_{2} \qquad ( 18 )$$

$${Y}_{2} = the Y position within the light - sharing segment \#2 = [ ( {E}_{I , 2} + {E}_{II , 2} ) - ( {E}_{III , 2} + {E}_{IV , 2} ) ] / {E}_{2} \qquad ( 19 )$$

$${R}_{2} = the DOI index within the light - sharing segment \#2 = max ( {E}_{I , 2} , {E}_{II , 2} {E}_{III , 2} {E}_{IV , 2} ) / {E}_{2} \qquad ( 20 )$$

Similarly, for the light-sharing segment #6, the following signals can be calculated:

$${E}_{6} = the total energy signal in light - sharing segment \#6 = {E}_{I , 6} + {E}_{II , 6} + {E}_{III , 6} + {E}_{IV , 6} \qquad ( 21 )$$

$${X}_{6} = the X position within the light - sharing segment \#6 = [ ( {E}_{II , 6} + {E}_{IV , 6} ) - ( {E}_{I , 6} + {E}_{III , 6} ) ] / {E}_{6} \qquad ( 22 )$$

$${Y}_{6} = the Y position within the light - sharing segment \#6 = [ ( {E}_{I , 6} + {E}_{II , 6} ) - ( {E}_{III , 6} + {E}_{IV , 6} ) ] / {E}_{6} \qquad ( 23 )$$

$${R}_{6} = the DOI index within the light - sharing segment \#6 = max ( {E}_{I , 6} , {E}_{II , 6} {E}_{III , 6} {E}_{IV , 6} ) / {E}_{6} \qquad ( 24 )$$

Additionally, the time stamp for the event can be calculated using an energy-weighting combination approach as:

$${T}_{weighted} = ( {E}_{I} * {T}_{I} + {E}_{II} * {T}_{II} + {E}_{III} * {T}_{III} + {E}_{IV} * {T}_{IV} ) / ( {E}_{I} + {E}_{II} + {E}_{III} + {E}_{IV} ) \qquad ( 25 )$$

In a scenario where an event deposited all its energy within one light-sharing segment (e.g., #2), Anger logic processing can be conducted to calculate energy, position (in 3-D; including a DOI index), and timing signals as below:

$${E}_{2} = the total energy signal in light - sharing segment \#2 = {E}_{I , 2} + {E}_{II , 2} + {E}_{III , 2} + {E}_{IV , 2} \qquad ( 26 )$$

$${X}_{2} = the X position within the light - sharing segment \#2 = [ ( {E}_{II , 2} + {E}_{IV , 2} ) - ( {E}_{I , 2} + {E}_{III , 2} ) ] / {E}_{2} \qquad ( 27 )$$

$${Y}_{2} = the Y position within the light - sharing segment \#2 = [ ( {E}_{I , 2} + {E}_{II , 2} ) - ( {E}_{III , 2} + {E}_{IV , 2} ) ] / {E}_{2} \qquad ( 28 )$$

$${R}_{2} = the DOI index within the light - sharing segment \#2 = max ( {E}_{I , 2} , {E}_{II , 2} {E}_{III , 2} {E}_{IV , 2} ) / {E}_{2} \qquad ( 29 )$$

$${T}_{weighted} = ( {E}_{I} * {T}_{I} + {E}_{II} * {T}_{II} + {E}_{III} * {T}_{III} + {E}_{IV} * {T}_{IV} ) / ( {E}_{I} + {E}_{II} + {E}_{III} + {E}_{IV} ) \qquad ( 30 )$$

In a scenario where an event deposited all its energy within three light-sharing segment (e.g., #2, #6, and #7), similar Anger logic processing can be conducted to calculate energy, position (in 3-D; including a DOI index), and timing signals as below:

$${E}_{2} = the total energy signal in light - sharing segment \#2 = {E}_{I , 2} + {E}_{II , 2} + {E}_{III , 2} + {E}_{IV , 2} \qquad ( 31 )$$

$${X}_{2} = the X position within the light - sharing segment \#2 = [ ( {E}_{II , 2} + {E}_{IV , 2} ) - ( {E}_{I , 2} + {E}_{III , 2} ) ] / {E}_{2} \qquad ( 32 )$$

$${Y}_{2} = the Y position within the light - sharing segment \#2 = [ ( {E}_{I , 2} + {E}_{II , 2} ) - ( {E}_{III , 2} + {E}_{IV , 2} ) ] / {E}_{2} \qquad ( 33 )$$

$${R}_{2} = the DOI index within the light - sharing segment \#2 = max ( {E}_{I , 2} , {E}_{II , 2} {E}_{III , 2} {E}_{IV , 2} ) / {E}_{2} \qquad ( 34 )$$

$${E}_{6} = the total energy signal in light - sharing segment \#6 = {E}_{I , 6} + {E}_{II , 6} + {E}_{III , 6} + {E}_{IV , 6} \qquad ( 35 )$$

$${X}_{6} = the X position within the light - sharing segment \#6 = [ ( {E}_{II , 6} + {E}_{IV , 6} ) - ( {E}_{I , 6} + {E}_{III , 6} ) ] / {E}_{6} \qquad ( 36 )$$

$${Y}_{6} = the Y position within the light - sharing segment \#6 = [ ( {E}_{I , 6} + {E}_{II , 6} ) - ( {E}_{III , 6} + {E}_{IV , 6} ) ] / {E}_{6} \qquad ( 37 )$$

$${R}_{6} = the DOI index within the light - sharing segment \#6 = max ( {E}_{I , 6} , {E}_{II , 6} {E}_{III , 6} {E}_{IV , 6} ) / {E}_{6} \qquad ( 38 )$$

$${E}_{7} = the total energy signal in light - sharing segment \#7 = {E}_{I , 7} + {E}_{II , 7} + {E}_{III , 7} + {E}_{IV , 7} \qquad ( 39 )$$

$${X}_{7} = the X position within the light - sharing segment \#7 = [ ( {E}_{II , 7} + {E}_{IV , 7} ) - ( {E}_{I , 7} + {E}_{III , 7} ) ] / {E}_{7} \qquad ( 40 )$$

$${Y}_{7} = the Y position within the light - sharing segment \#7 = [ ( {E}_{I , 7} + {E}_{II , 7} ) - ( {E}_{III , 7} + {E}_{IV , 7} ) ] / {E}_{7} \qquad ( 41 )$$

$${R}_{7} = the DOI index within the light - sharing segment \#7 = max ( {E}_{I , 7} , {E}_{II , 7} {E}_{III , 7} {E}_{IV , 7} ) / {E}_{7} \qquad ( 42 )$$

$${T}_{weighted} = ( {E}_{I} * {T}_{I} + {E}_{II} * {T}_{II} + {E}_{III} * {T}_{III} + {E}_{IV} * {T}_{IV} ) / ( {E}_{I} + {E}_{II} + {E}_{III} + {E}_{IV} ) \qquad ( 43 )$$

Then, various correction methods can be implemented on the calculated energy, position, and timing signals, including, but not limited to, converting the DOI index to DOI information. For example, based on a mathematical model representing the exponential decay of gamma interactions in the depth direction, the DOI index can be converted to the actual depth within the crystal. During a calibration phase, a radiation source can be placed on the side of the detector module, so that the DOI index can be calibrated at different depths within the crystals. Alternatively, background radiation from radioactive Lu-176 in the scintillator material can be used during calibration to determine a conversion from DOI index to depth which results in the most uniform distribution of depths for background radiation events.

For some readout methods, such as the leading-edge-threshold method, the timing signals (T_{I}, T_{II}, T_{III}, T_{IV}) could depend on the energy levels (E_{I}, E_{II}, E_{III}, E_{IV}). In this case, a correction can be applied to the timing signals based upon energies before applying the above equations (17)-(25), (26)-(30), or (31)-(43), for example.

In another example, before applying the above equations, the nonlinear energy response, which could be due to non-proportionality in the scintillation response of the crystals, the nonlinear response of the photosensors (such as SiPMs), and the nonlinearity of the electronics readout methods, can be corrected before applying the above equations.

Note that while the implementation described above determines the 3D position, including position information in the X, Y, and Z (DOI) directions, the concepts of the present disclosure are also applicable to non-DOI detectors.

As an alternative approach to the Anger-like positioning scheme, the signal levels determined at the SiPMs can be input into a neural network trained to infer the 3-D position of the first interaction during the event, the overall energy deposited, etc. The neural network can also incorporate various corrections, such as non-linearity corrections.

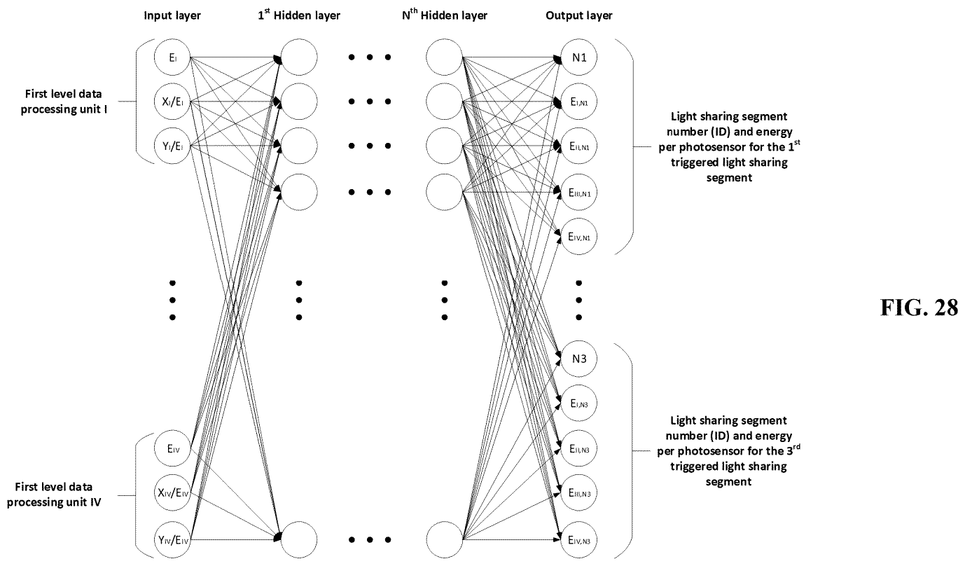

Moreover, machine learning techniques can replace LUTs to implement the approaches described above. FIG. 28 shows an exemplary structure of a single neutral network that infers the light-sharing segment number(s) (ID(s)) of the light-sharing segment(s) involved in an event and signal levels at SiPMs within the involved light-sharing segment(s), in accordance with embodiments of the present disclosure. The neural network receives the digital outputs Eₘ, Xₘ, and Yₘ from the first-level electronic units as inputs, where m=I, II, III, and IV. The outputs of the neural network include the segment numbers (IDs) and the signal levels at the SiPMs of the three light-sharing segments with the most energy deposited. Referring back to FIG. 11, the steps S1110-S1120 in this situation can be replaced by a neural network inference process. Accordingly, the offline calibration procedure 1150 in FIG. 11 can be replaced with a neural network training procedure.

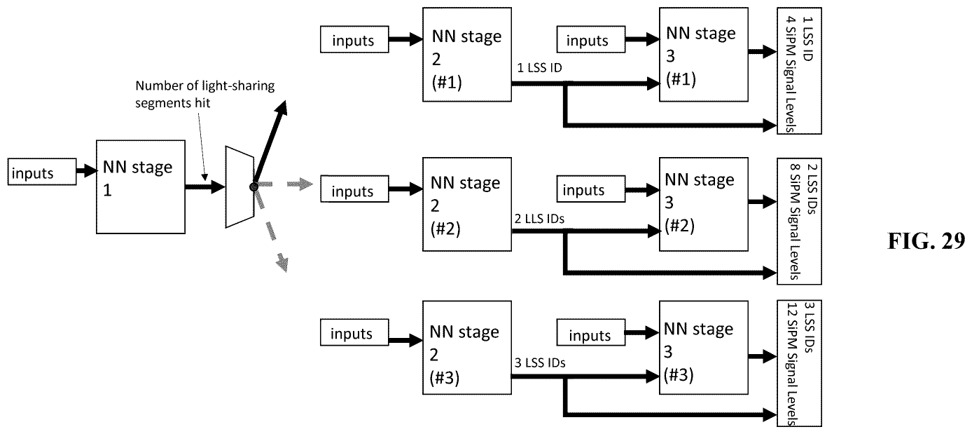

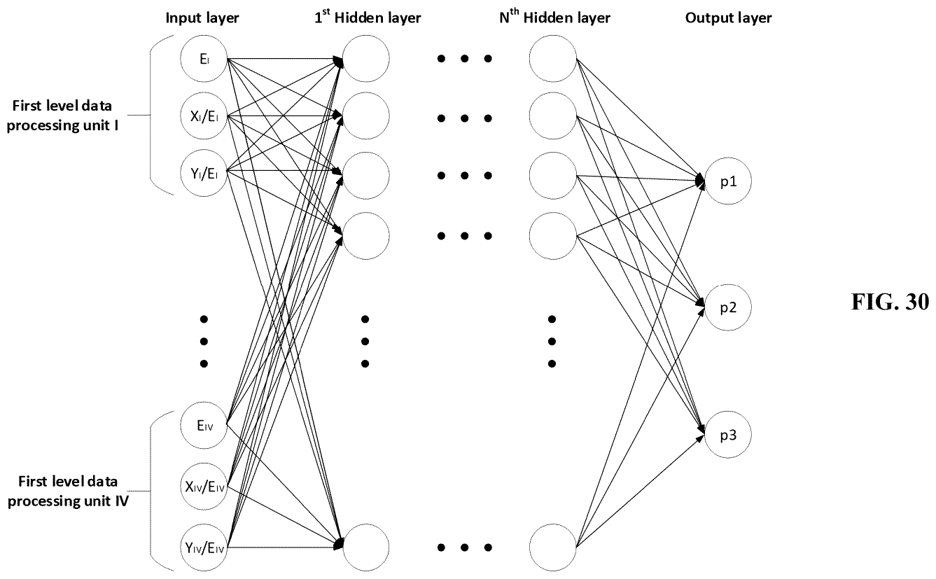

FIG. 29 shows a high-level view of the architecture of a multi-stage neural network that infers the light-sharing segment number(s) (ID(s)) of the light-sharing segment(s) involved in an event and the signal levels at the SiPMs within the involved light-sharing segment(s), in accordance with embodiments of the present disclosure. The architecture includes three stages. The first stage determines how many light-sharing segments were hit during the event. Based on the output of the first stage, the path for the subsequent stages can be determined. Three separate second stages identify which specific segment(s) among the 16 light-sharing segments was hit. Three separate third stages determines the signal levels at 4, 8, or 12 SiPMs within the hit light-sharing segment(s). Further details regarding the neural networks used in each of the three stages can be found in FIGS. 30-32C.

FIG. 30 shows an exemplary structure of the first stage of the multi-stage neural network shown in FIG. 29, in accordance with embodiments of the present disclosure. For example, a classifier neural network can be used to determine the number of light-sharing segments hit during the event. The outputs of this neural network include probabilities that one, two, and three light-sharing segments were hit. The final number of the light-sharing segments hit, i.e., 1, 2, or 3, is determined based on which probably has the highest value.

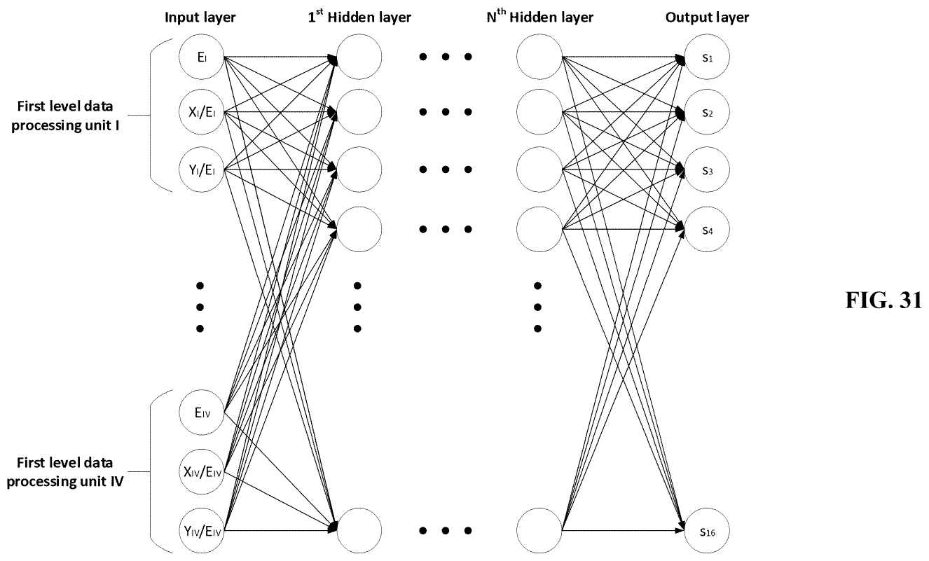

FIG. 31 shows an exemplary structure of the second stage of the multi-stage neural network shown in FIG. 29, in accordance with embodiments of the present disclosure. Based on the output from the first stage, one of three classifier neural networks corresponding to 1, 2, or 3 light-sharing segments can be chosen to determine the segment number(s) (ID(s)). The three classifier neutral networks can have the same general structure as shown in FIG. 31. The outputs include probabilities that each of the 16 light-sharing segments was hit. The specific ID of the single light-sharing segment hit can be determined by the classifier neural network corresponding to 1 light-sharing segment by identifying the segment with the highest probability. Similarly, the specific IDs of the two light-sharing segments hit can be determined by the classifier neural network corresponding to 2 light-sharing segment by identifying the segments with the two highest probabilities. The specific IDs of the three light-sharing segments hit can be determined by the classifier neural network corresponding to 3 light-sharing segment by identifying the segments with the three highest probabilities.

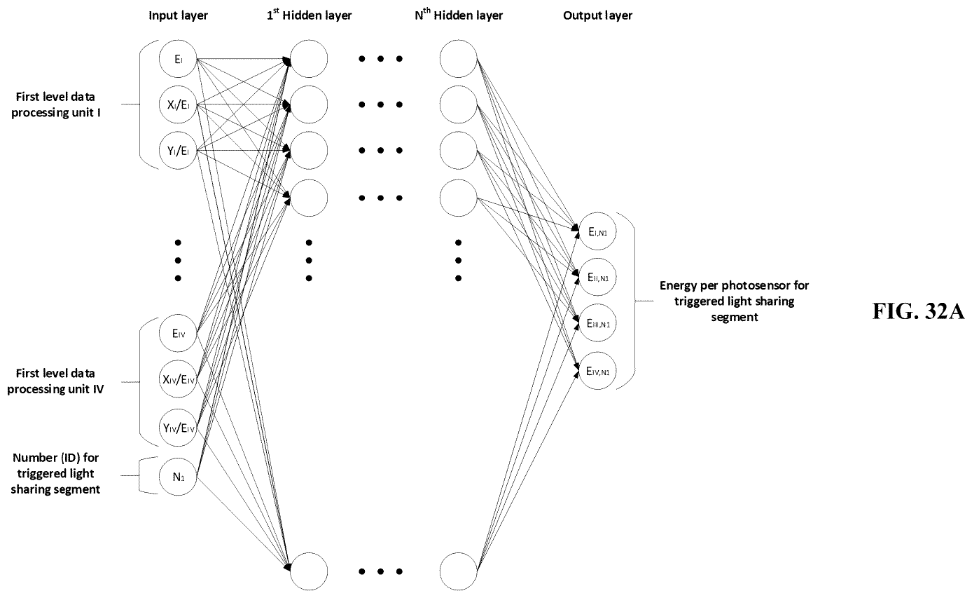

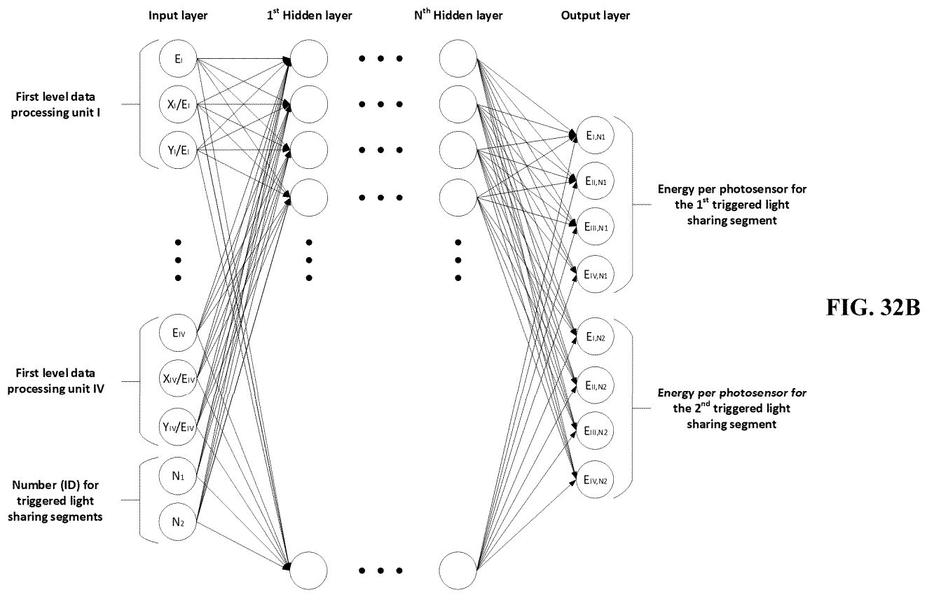

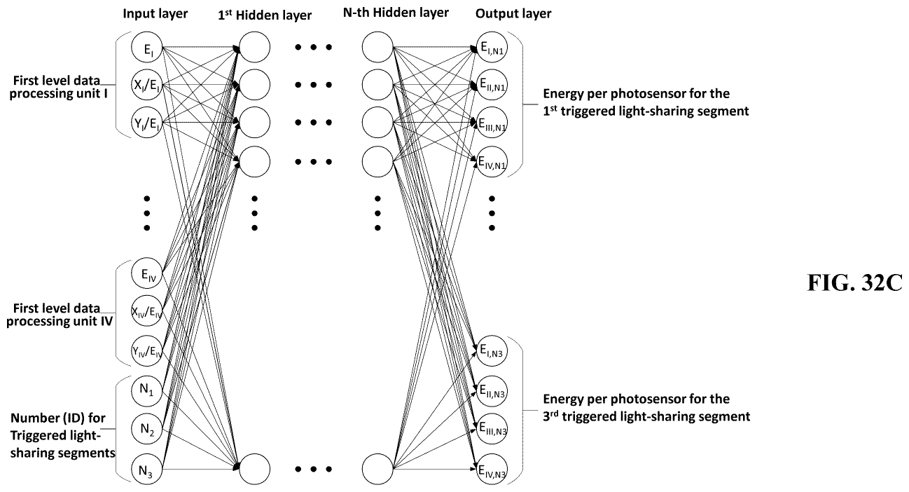

FIGS. 32A-32C show exemplary structures of the third stage of the multi-stage neural network shown in FIG. 29, in accordance with embodiments of the present disclosure. Based on the output from the first stage, one of three neutral networks corresponding to 1, 2, or 3 light-sharing segments can be chosen to determine the signal levels at each SiPM within the hit light-sharing segment(s). In the scenario where one light-sharing segment was hit, the neural network shown in FIG. 32A determines the signal levels at each of the 4 SiPMs within that single light-sharing segment. In the scenario where two light-sharing segments were hit, the neural network shown in FIG. 32B determines the signal levels at each of the 8 SiPMs within these two light-sharing segments. In the scenario where three light-sharing segments were hit, the neural network shown in FIG. 32C determines the signal levels at each of the 12 SiPMs within these three light-sharing segments.

The approaches described in FIGS. 28-32C are aimed at determination of the signal level at each individual photosensor. For imaging applications, the final information of interest from a detected event is the interaction position of the first interaction in the detector, which can be described by Xtal_{final}, identifying the crystal in which the first interaction occurred, and DOI_{final}, identifying the depth of the first interaction. Particularly in time-of-flight PETs, the interaction time of the first interaction, including corrections for effects such as signal-amplitude-dependent time-talk, is also required, along with a corrected total energy of the event which can be used to eliminate many of the events which underwent scatter in the patient. A neural network approach can be used to convert the signal level at each individual photosensor to a 3-D position of the first interaction, a corrected time for the first interaction, and a total energy for the event.

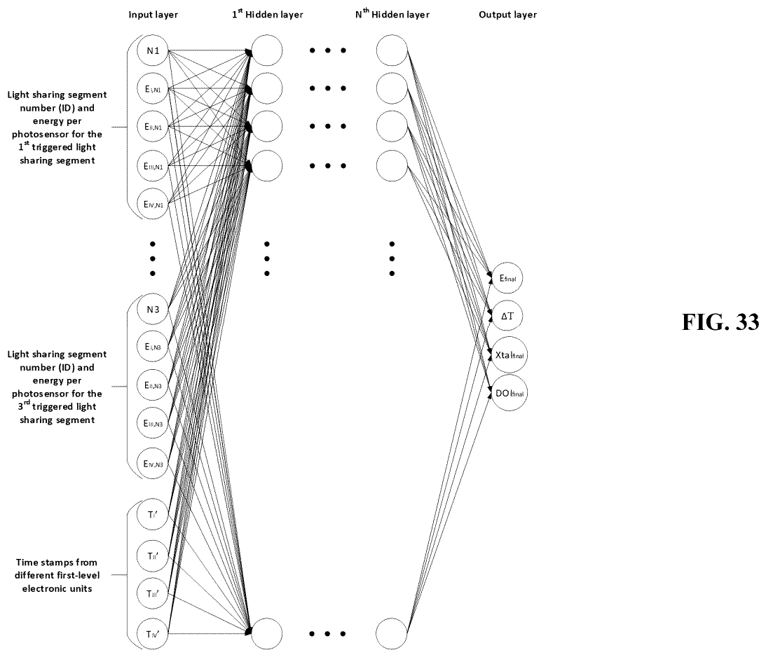

FIG. 33 shows a high-level view of the architecture of a neural network to estimate the final energy, position and time stamp of the gamma event. The inputs to the neutral network include: N1, E_{I-IV,N1}; N2, E_{I-IV,N2}; N3, E_{I-IV,N3}; all of which are outputs from the neural networks in FIGS. 28-32C. For events that only trigger 1 or 2 light-sharing segments, the values of the undetermined inputs can be set to 0. Also included as inputs to the neural network are relative times T′_{I-IV}. A process for calculating the relative times is described below. Relative times are used in the neural network approach, rather than absolute times, because absolute times are unbounded (i.e. they have no maximum value) and neural networks generally perform best when the inputs and outputs are well bounded (i.e. always occur within a known range and/or have a well-defined variance).

For approaches based on neural networks or other machine learning techniques, the choice of the training data is important. Simulated data can be used for training in embodiments of the disclosure. For example, simulations can include gamma transport and energy deposition, optical transport, a detailed model of the SiPM signal detection process (including dark counts and cross-talk), and a model of the first-level electronic units. Before using the simulated data in the training process, simulation parameters can be validated and tuned using measured detector performance to achieve better agreement. Importantly, for the training of the network shown in FIG. 33, when using simulated data, the first interaction position, Xtal_{final} and DOI_{final}, and the precise time of the first interaction, To, are known and can be used as target results for the training of the neural network.

As mentioned above, relative times are used as the input to the network in FIG. 33. For relative times, a reference time for each event is chosen and this reference time is subtracted from each of the measure time stamps. One appropriate choice for a reference time is T_{weighted}, as defined in equation 43. The relative time stamp is then calculated by T′_{I,II,III,IV}=T_{I,II,III,IV}−T_{weighted}, and the training target time is calculated by ΔT=T₀−T_{weighted}. After inference using the neural network, the final time stamp (absolute time) is calculated as T_{final}=T_{weighted}+ΔT. Other appropriate choices for the event reference time include the earliest of T_{I-IV} or a simple mean of T_{I-IV}.

The neural network described above with reference to FIG. 33 can also be applied to the results from the LUT approach to determining the signal level on each individual photosensor, in order to determine the final energy, position and time stamp of the gamma event.

Alternatively, a neural network or a number of neural networks can be trained to directly infer the set of energy and 3-D position signals and the time stamp signal of the event based on the digital output signals (i.e., T-signals, E-signals, X-signals, and Y-signals) acquired from the first-level electronic units. This approach eliminates the need for explicit determination of the signal level on each individual photosensor.

### Detector Design 2

Those skilled in the art will appreciate that the number (N) of photosensors covered by one first-level electronic unit and the number (M) of first-level electronic units covered by one second-level electronic unit can be different from those of the Detector Design 1.

In this exemplary DOI TOF detector design, each detector module includes a 36×36 crystal array coupled to an 18×18 photosensor array. That is, each 2×2 crystal array is coupled to a single photosensor. The 18×18 photosensor array is readout by nine first-level electronic units and one second-level electronic unit. Segmented light sharing is implemented within 3×3 photosensor arrays, forming 36 light-sharing segments.

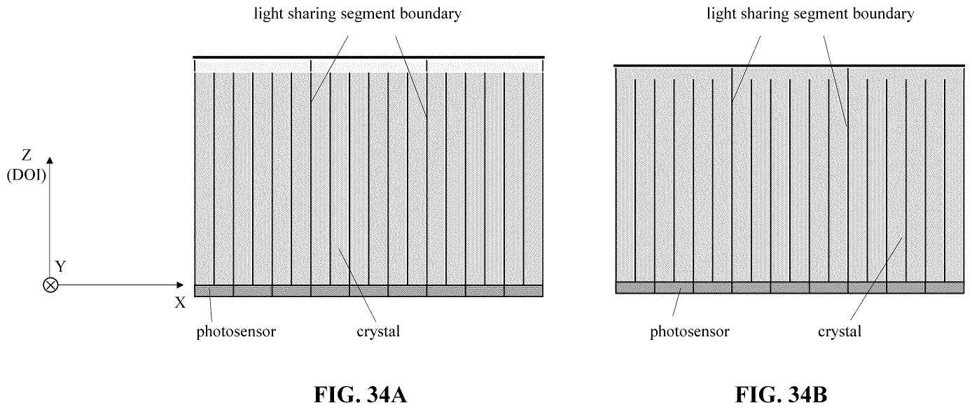

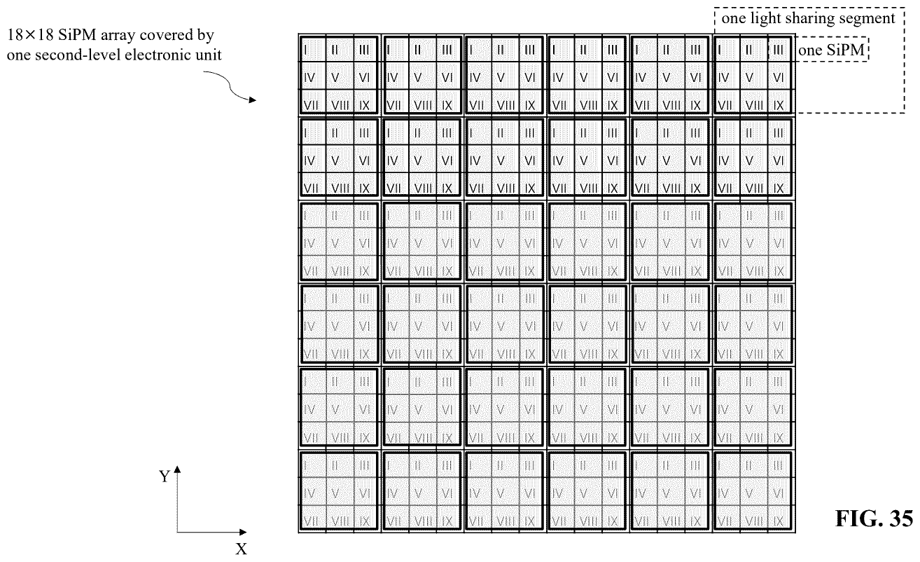

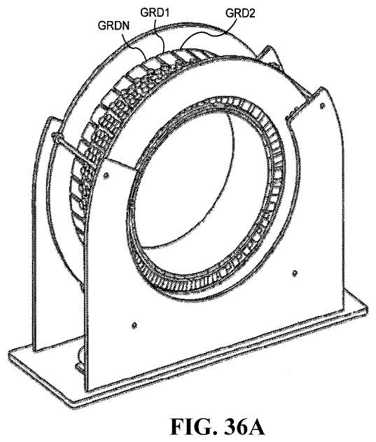

FIG. 34A shows one embodiment where segmented light sharing is achieved within a 3×3 photosensor array, through a light guide. FIG. 34B shows one embodiment where segmented light sharing is achieved within a 3×3 photosensor array, using partial reflectors and partial optical glue between crystals. FIG. 35 illustrates the connection between the photosensors and the first-level electronic units I-IX. The nine photosensors within each 3×3 light-sharing segment are connected to the nine first-level electronic units in a one-to-one manner. Consequently, each first-level electronic unit receives outputs from 36 photosensors located in 36 different light-sharing segments, resulting in an input multiplexing ratio of 36. Each first-level electronic unit has one readout channel (T) for timing and three readout channels (E, X, Y) for energy and position.

Note that Detector Design 1 is preferable to Detector Design 2. Due to the smaller light-sharing segment used in Detector Design 1, more multiple-hit events generate hits in two light-sharing segments, making it more likely to obtain independent X, Y, and DOI positions for these hits. This increases the likelihood of identifying the “first hit.” Moreover, as fewer photosensors are read by one first-level electronic unit in Detector Design 1, the impact from pile-up and the timing resolution degradation due to more dark counts and increased capacitance are reduced.

With the exemplary multiplexing schemes and segmented light sharing described in the disclosure, the signal level in each photosensor can be determined without requiring a separate read-out channel for each photosensor. The DOI can be decoded, and a large fraction of inter-crystal Compton scatter events can be resolved into separate discrete hits. There is no timing performance degradation caused by layered crystals of different materials or varying crystal layer shape. Additionally, no specialized light guide or extra photosensor/electronic channels are required, resulting in a low manufacturing cost. The power consumption of the electronics in this multiplexing scheme is also comparable to that of conventional multiplexing schemes.

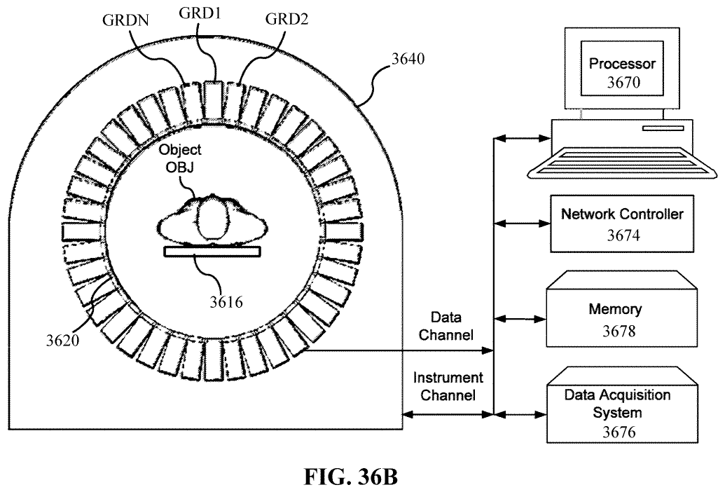

FIGS. 36A and 36B illustrate in implementation in which a medical imaging system includes a PET scanner that can implement the methods described in this disclosure. The PET scanner includes a plurality of gamma-ray detectors (GRDs) (e.g., GRD1, GRD2, through GRDN) that are each configured as rectangular detector modules.

Each GRD can include a two-dimensional array of individual detector crystals, which absorb gamma radiation and emit scintillation photons. The scintillation photons can be detected by a two-dimensional array of photodetectors or photosensors, e.g., photomultiplier tubes (PMTs), silicon photomultipliers (SiPMs), etc. A light guide can either be disposed between the array of detector crystals and the photodetectors or on the opposite end of the crystal array from the photodetectors.

Each photodetector (e.g., SiPM or PMT) can produce an analog signal that indicates when scintillation events occur, and an energy of the gamma-ray producing the detection event. Moreover, the photons emitted from one detector crystal can be detected by more than one photodetector, and, based on the analog signal produced at each photodetector, the detector crystal corresponding to the detection event can be determined using various methods, including, but not limited to, the multiplexing and analysis scheme described above, Anger logic and crystal decoding.

FIG. 36B shows one example of the arrangement of the PET scanner, in which the object OBJ to be imaged rests on a table 3616 and the GRD modules GRD1 through GRDN are arranged circumferentially around the object OBJ and the table 3616. The GRDs can be fixedly connected to a circular component 3620 that is fixedly connected to a gantry 3640. The gantry 3640 houses many parts of the PET scanner. The gantry 3640 of the PET scanner also includes an open aperture through which the object OBJ and the table 3616 can pass, and gamma-rays emitted in opposite directions from the object OBJ due to an annihilation event can be detected by the GRDs and timing and energy information can be used to determine coincidences for gamma-ray pairs.

In FIG. 36B, circuitry and hardware are also shown for acquiring, storing, processing, and distributing gamma-ray detection data. The circuitry and hardware include: a processor 3670, a network controller 3674, a memory 3678, and a data acquisition system (DAS) 3676. The PET scanner also includes a data channel that routes detection measurement results from the GRDs to the DAS 3676, the processor 3670, the memory 3678, and the network controller 3674. The data acquisition system 3676 can control the acquisition, digitization, and routing of the detection data from the detectors. In one implementation, the DAS 3676 controls the movement of the bed 3616. The processor 3670 performs functions including reconstructing images from the detection data, pre-reconstruction processing of the detection data, and post-reconstruction processing of the image data, as discussed herein.

The processor 3670 can be configured to perform various steps of the methods described herein and variations thereof. The processor 3670 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 3570 can execute a computer program including a set of computer-readable instructions that perform various steps of the described methods, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 3678 can be a hard disk drive, CD-ROM drive, DVD drive, FLASH drive, RAM, ROM or any other electronic storage known in the art.

The network controller 3674, such as an Intel Ethernet PRO network interface card from Intel Corporation of America, can interface between the various parts of the PET scanner. Additionally, the network controller 3674 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Although the above descriptions are provided in the context of PET scanners with DOI capacities, those skilled in the art will recognize that the disclosed concepts can be applied to other medical imaging systems based on gamma-ray detection, such as single-photon emission computed tomography (SPECT) scanners, with or without DOI capacities.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.

(1) A medical imaging apparatus for gamma-ray detection, comprising: a detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2; and circuitry including (1) a plurality of sets of M first-level electronic units and (2) a second-level electronic unit, each set of M first-level electronic units being configured to read the electrical signals generated by the photosensor array of a corresponding one of the plurality of detector modules, wherein for each detector module of the plurality of detector modules, each first-level electronic unit of a corresponding set of M first-level electronic units reads the electrical signals generated by N photosensors, in a multiplexing manner, to generate timing, energy, and position readout signals on a single set of readout channels, the second-level electronic unit processes the timing, energy, and position readout signals output from M sets of readout channels of the set of M first-level electronic units, with respect to each first-level electronic unit of the set of M first-level electronic units, each of the N photosensors read by the first-level electronic unit is situated in a corresponding different one of the N light-sharing segments, and with respect to each light-sharing segment of the N light-sharing segments, each of the M photosensors covered by the light-sharing segment is read by a corresponding different first-level electronic unit of the set of M first-level electronic units.

(2) The medical imaging apparatus of (1), wherein the second-level electronic unit is further configured to: upon the set of M first-level electronic units being triggered by a gamma-ray detection event, acquire the timing, energy, and position readout signals from the set of M first-level electronic units, identify a particular light-sharing segment within which energy was deposited by gamma-ray interactions occurring during the gamma-ray detection event, based on the acquired energy and position readout signals, and determine, based on the acquired energy, position, and timing readout signals, a set of energy, position, and timing signals with respect to the gamma-ray interactions occurring during the gamma-ray detection event.

(3) The medical imaging apparatus of (2), wherein each light-sharing segment of the N light-sharing segments is configured to encode depth of interaction (DOI) information of gamma-ray interactions occurring therewithin, the second-level electronic unit is further configured to determine a 3D position signal, as the position signal with respect to the gamma-ray interactions occurring during the gamma-ray detection event, and the 3D position signal includes position information of the gamma-ray interactions occurring during the gamma-ray detection event, in a depth direction of the scintillation crystals.

(4) The medical imaging apparatus of (2), wherein the particular light-sharing segment includes up to three involved light-sharing segments within which energy was deposited by the gamma-ray interactions occurring during the gamma-ray detection event, and the second-level electronic unit is further configured to: for each first-level electronic unit of the set of M first-level electronic units, calculate a pair of measured positions based on the acquired energy and position readout signals, use the calculated pairs of measured positions to identify the up to three involved light-sharing segments, based on a particular look-up table from a pre-prepared set of look-up tables, each look-up table corresponding to a probable segment-combination of up to three light-sharing segments within which energy is deposited by gamma-ray interactions occurring during a gamma-ray detection event, determine a signal level at each photosensor within the identified up to three involved light-sharing segments, based on the calculated pairs of measured positions, peak positions in the particular look-up table that represent positions of corresponding single-light-sharing-segment gamma-ray detection events, and the acquired energy readout signals, and determine the set of energy, position, and timing signals, based on the determined signal levels at the photosensors within the identified up to three involved light-sharing segments and the acquired energy and timing readout signals.

(5) The medical imaging apparatus of (4), wherein the second-level electronic unit is further configured to, during a calibration phase: upon the set of M first-level electronic units being triggered by each gamma-ray detection event of a plurality of gamma-ray detection events, collect the timing, energy, and position readout signals from the set of M first-level electronic units, generate a flood histogram for each first-level electronic unit of the set of M first-level electronic units, the flood histogram representing a 2D position map of the plurality of gamma-ray detection events detected by the first-level electronic unit, generate a set of look-up tables for each first-level electronic unit of the set of M first-level electronic units, based on the flood histogram generated for the first-level electronic unit, and save the generated sets of look-up tables, as the pre-prepared set of look-up tables.

(6) The medical imaging apparatus of (4), wherein the second-level electronic unit is further configured to determine the set of energy, position, and timing signals by performing Anger logic calculations based on the determined signal levels at the photosensors within the identified up to three involved light-sharing segments to determine the energy and position signals, and performing weighting calculation based on the acquired energy and timing readout signals to determine the timing signal, or inputting the determined signal levels at the photosensors within the identified up to three involved light-sharing segments and the acquired energy and timing readout signals into a neural network, and obtaining an output of the neural network as the determined set of energy, position, and timing signals, where the neural network has been trained during a calibration phase to establish a mapping relationship from signal levels at photosensors within up to three light-sharing segments involved in a gamma-ray detection event and energy and timing readout signals acquired for the gamma-ray detection event to energy, position, and timing signals with respect to gamma-ray interactions occurring during the gamma-ray detection event.

(7) The medical imaging apparatus of (4), wherein the second-level electronic unit is further configured to identify the up to three involved light-sharing segments by: for each first-level electronic unit of the set of M first-level electronic units, with respect to a voting region with a size dependent on the acquired energy readout signal, casting votes for the pre-prepared set of look-up tables, based on the calculated pair of measured positions, selecting, as the particular look-up table, a specific look-up table from the pre-prepared set of look-up tables that receives a most number of votes from the set of M first-level electronic units, and determining, the up to three light-sharing segments included in the segment-combination corresponding to the specific look-up table, as the identified up to three involved light-sharing segments.

(8) The medical imaging apparatus of (7), wherein each look-up table of the pre-prepared look-up tables has a probability ranking that represents likelihood of the corresponding segment-combination among all of the probable segment-combinations, and the second-level electronic unit is further configured to: when two or more look-up tables receive the most number of votes from the set of M first-level electronic units, select, based on the respective probability rankings, one of the two or more look-up tables that has higher likelihood, as the particular look-up table, when two or more look-up tables receive the most number of votes from the set of M first-level electronic units, have a same probability ranking, and correspond to two-light-sharing-segment-combinations, select one of the two or more look-up tables that a vector connecting peak positions representing single-light-sharing segment gamma-ray detection events corresponding to the two light-sharing segments has a better match with a vector representing a linear fit to the measured positions, as the particular look-up table, and when two or more look-up tables receive the most number of votes from the set of M first-level electronic units, have a same probability ranking, and correspond to three-light-sharing-segment-combinations, and the two three-light-sharing-segment-combinations have a common light-sharing segment, select one of the two or more look-up tables that has more number of votes from the set of M first-level electronic units that do not vote for a look-up table corresponding to a single-light-sharing-segment-combination of the common light-sharing segment, as the particular look-up table.

(9) The medical imaging apparatus of (2), wherein the particular light-sharing segment includes up to three involved light-sharing segments within which energy was deposited by the gamma-ray interactions occurring during the gamma-ray detection event, and the second-level electronic unit is further configured to: input the acquired energy and position readout signals into a neural network, and obtain, at an output of the neural network, segment identifiers of the up to three involved light-sharing segments and a signal level at each photosensor within the up to three involved light-sharing segments, where the neural network has been trained during a calibration phase to establish a mapping relationship from (1) energy and position readout signals acquired for a gamma-ray detection event to (2) up to three light-sharing segments involved in the gamma-ray detection event and signal levels at photosensors within the up to three light-sharing segments involved in the gamma-ray detection event.

(10) The medical imaging apparatus of (1), wherein each detector module is divided into 16 light-sharing segments, and each light-sharing segment covers 4 photosensors of the photosensor array, or each detector module is divided into 36 light-sharing segments, and each light-sharing segment covers 9 photosensors of the photosensor array.

(11) A method for reading and processing electric signals in a medical imaging apparatus for gamma-ray detection, the medical imaging apparatus including a detector and circuitry, the detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2, the circuitry including (1) a plurality of sets of M first-level electronic units and (2) a second-level electronic unit, each set of M electronic units being configured to read the electrical signals generated by the photosensor array of a corresponding one of the plurality of detector modules, the method comprising, for each detector module of the plurality of detector modules: reading via each first-level electronic unit of a corresponding set of M first-level electronic units, the electrical signals generated by N photosensors in a multiplexing manner to generate timing, energy, and position readout signals on a single set of readout channels; and processing via the second-level electronic unit, the timing, energy, and position readout signals output from M sets of readout channels of the set of M first-level electronic units, wherein with respect to each first-level electronic unit of the set of M first-level electronic units, each of the N photosensors read by the first-level electronic unit is situated in a corresponding different one of the N light-sharing segments, and with respect to each light-sharing segment of the N light-sharing segments, each of the M photosensors covered by the light-sharing segment is read by a corresponding different first-level electronic unit of the set of M first-level electronic units.

(12) The method of (11), wherein the processing step further comprises: upon the set of M first-level electronic units being triggered by a gamma-ray detection event, acquiring the timing, energy, and position readout signals from the set of M first-level electronic units, identifying a particular light-sharing segment within which energy was deposited by gamma-ray interactions occurring during the gamma-ray detection event, based on the acquired energy and position readout signals, and determining, based on the acquired energy, position, and timing readout signals, a set of energy, position, and timing signals with respect to the gamma-ray interactions occurring during the gamma-ray detection event.

(13) The method of (12), wherein each light-sharing segment of the N light-sharing segments is configured to encode depth of interaction (DOI) information of gamma-ray interactions occurring therewithin, the determining step further comprises determining a 3D position signal, as the position signal with respect to the gamma-ray interactions occurring during the gamma-ray detection event, and the 3D position signal includes position information of the gamma-ray interactions occurring during the gamma-ray detection event, in a depth direction of the scintillation crystals.

(14) The method of (12), wherein the particular light-sharing segment includes up to three involved light-sharing segments within which energy was deposited by the gamma-ray interactions occurring during the gamma-ray detection event, and the steps of identifying and determining further comprise: for each first-level electronic unit of the set of M first-level electronic units, calculating a pair of measured positions based on the acquired energy and position readout signals, using the calculated pairs of measured positions to identify the up to three involved light-sharing segments, based on a particular look-up table from a pre-prepared set of look-up tables, each look-up table corresponding to a probable segment-combination of up to three light-sharing segments within which energy is deposited by gamma-ray interactions occurring during a gamma-ray detection event, determining a signal level at each photosensor within the identified up to three involved light-sharing segments, based on the calculated pairs of measured positions, peak positions in the particular look-up table that represent positions of corresponding single-light-sharing-segment gamma-ray detection events, and the acquired energy readout signals, and determining the set of energy, position, and timing signals, based on the determined signal levels at the photosensors within the identified up to three involved light-sharing segments and the acquired energy and timing readout signals.

(15) The method of (14), further comprises, during a calibration phase: upon the set of M first-level electronic units being triggered by each gamma-ray detection event of a plurality of gamma-ray detection events, collecting the timing, energy, and position readout signals from the set of M first-level electronic units, generating a flood histogram for each first-level electronic unit of the set of M first-level electronic units, the flood histogram representing a 2D position map of the plurality of gamma-ray detection events detected by the first-level electronic unit, generating a set of look-up tables for each first-level electronic unit of the set of M first-level electronic units, based on the flood histogram generated for the first-level electronic unit, and saving the generated sets of look-up tables, as the pre-prepared set of look-up tables.

(16) The method of (14), wherein the step of determining the set of energy, position, and timing signals further comprises: performing Anger logic calculations based on the determined signal levels at the photosensors within the identified up to three involved light-sharing segments to determine the energy and position signals, and performing weighting calculation based on the acquired energy and timing readout signals to determine the timing signal, or inputting the determined signal levels at the photosensors within the identified up to three involved light-sharing segments and the acquired energy and timing readout signals into a neural network, and obtaining an output of the neural network as the determined set of energy, position, and timing signals, where the neural network has been trained during a calibration phase to establish a mapping relationship from signal levels at photosensors within up to three light-sharing segments involved in a gamma-ray detection event and energy and timing readout signals acquired for the gamma-ray detection event to energy, position, and timing signals with respect to gamma-ray interactions occurring during the gamma-ray detection event.

(17) The method of (14), wherein the step of using the calculated pairs of measured positions to identify the up to three involved light-sharing segments further comprises: for each first-level electronic unit of the set of M first-level electronic units, with respect to a voting region with a size dependent on the acquired energy readout signal, casting votes for the pre-prepared set of look-up tables, based on the calculated pair of measured positions, selecting, as the particular look-up table, a specific look-up table from the pre-prepared set of look-up tables that receives a most number of votes from the set of M first-level electronic units, and determining, the up to three light-sharing segments included in the segment-combination corresponding to the specific look-up table, as the identified up to three involved light-sharing segments.

(18) The method of (17), wherein each look-up table of the pre-prepared look-up tables has a probability ranking that represents likelihood of the corresponding segment-combination among all of the probable segment-combinations, and the selecting step further comprises: when two or more look-up tables receive the most number of votes from the set of M first-level electronic units, selecting, based on the respective probability rankings, one of the two or more look-up tables that has higher likelihood, as the particular look-up table, when two or more look-up tables receive the most number of votes from the set of M first-level electronic units, have a same probability ranking, and correspond to two-light-sharing-segment-combinations, selecting one of the two or more look-up tables that a vector connecting peak positions representing single-light-sharing segment gamma-ray detection events corresponding to the two light-sharing segments has a better match with a vector representing a linear fit to the measured positions, as the particular look-up table, and when two or more look-up tables receive the most number of votes from the set of M first-level electronic units, have a same probability ranking, and correspond to three-light-sharing-segment-combinations, and the two three-light-sharing-segment-combinations have a common light-sharing segment, selecting one of the two or more look-up tables that has more number of votes from the set of M first-level electronic units that do not vote for a look-up table corresponding to a single-light-sharing-segment-combination of the common light-sharing segment, as the particular look-up table.

(19) The method of (12), wherein the particular light-sharing segment includes up to three involved light-sharing segments within which energy was deposited by the gamma-ray interactions occurring during the gamma-ray detection event, and the steps of identifying and determining further comprise: inputting the acquired energy and position readout signals into a neural network, and obtaining, at an output of the neural network, segment identifiers of the up to three involved light-sharing segments and a signal level at each photosensor within the up to three involved light-sharing segments, where the neural network has been trained during a calibration phase to establish a mapping relationship from (1) energy and position readout signals acquired for a gamma-ray detection event to (2) up to three light-sharing segments involved in the gamma-ray detection event and signal levels at photosensors within the up to three light-sharing segments involved in the gamma-ray detection event.

(20) A non-transitory computer readable medium having instructions stored therein that, when executed by one or more processors, cause the one or more processors to perform a method for reading and processing electric signals in a medical imaging apparatus for gamma-ray detection, the medical imaging apparatus including a detector and circuitry, the detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2, the circuitry including (1) a plurality of sets of M first-level electronic units and (2) a second-level electronic unit, each set of M first-level electronic units being configured to read the electrical signals generated by the photosensor array of a corresponding one of the plurality of detector modules, the method comprising, for each detector module of the plurality of detector modules: reading via each first-level electronic unit of a corresponding set of M first-level electronic units, the electrical signals generated by N photosensors in a multiplexing manner to generate timing, energy, and position readout signals on a single set of readout channels; and processing via the second-level electronic unit, the timing, energy, and position readout signals output from M sets of readout channels of the set of M first-level electronic units, wherein with respect to each first-level electronic unit of the set of M first-level electronic units, each of the N photosensors read by the first-level electronic unit is situated in a corresponding different one of the N light-sharing segments, and with respect to each light-sharing segment of the N light-sharing segments, each of the M photosensors covered by the light-sharing segment is read by a corresponding different first-level electronic unit of the set of M first-level electronic units.

Numerous modifications and variations of the embodiments presented herein are possible in light of the above teachings. It is therefore to be understood that within the scope of the claims, the disclosure may be practiced otherwise than as specifically described herein.

What is claimed is:

1. A medical imaging apparatus for gamma-ray detection, comprising:
a detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2; and
circuitry including (1) a plurality of sets of M first-level electronic units and (2) a second-level electronic unit, each set of M first-level electronic units being configured to read the electrical signals generated by the photosensor array of a corresponding one of the plurality of detector modules,
wherein for each detector module of the plurality of detector modules,
each first-level electronic unit of a corresponding set of M first-level electronic units reads the electrical signals generated by N photosensors, in a multiplexing manner, to generate timing, energy, and position readout signals on a single set of readout channels,
the second-level electronic unit processes the timing, energy, and position readout signals output from M sets of readout channels of the set of M first-level electronic units,
with respect to each first-level electronic unit of the set of M first-level electronic units, each of the N photosensors read by the first-level electronic unit is situated in a corresponding different one of the N light-sharing segments, and
with respect to each light-sharing segment of the N light-sharing segments, each of the M photosensors covered by the light-sharing segment is read by a corresponding different first-level electronic unit of the set of M first-level electronic units.

2. The medical imaging apparatus of claim 1, wherein the second-level electronic unit is further configured to:
upon the set of M first-level electronic units being triggered by a gamma-ray detection event, acquire the timing, energy, and position readout signals from the set of M first-level electronic units,
identify a particular light-sharing segment within which energy was deposited by gamma-ray interactions occurring during the gamma-ray detection event, based on the acquired energy and position readout signals, and
determine, based on the acquired energy, position, and timing readout signals, a set of energy, position, and timing signals with respect to the gamma-ray interactions occurring during the gamma-ray detection event.

3. The medical imaging apparatus of claim 2, wherein each light-sharing segment of the N light-sharing segments is configured to encode depth of interaction (DOI) information of gamma-ray interactions occurring therewithin,
the second-level electronic unit is further configured to determine a 3D position signal, as the position signal with respect to the gamma-ray interactions occurring during the gamma-ray detection event, and
the 3D position signal includes position information of the gamma-ray interactions occurring during the gamma-ray detection event, in a depth direction of the scintillation crystals.

4. The medical imaging apparatus of claim 2, wherein the particular light-sharing segment includes up to three involved light-sharing segments within which energy was deposited by the gamma-ray interactions occurring during the gamma-ray detection event, and the second-level electronic unit is further configured to:
for each first-level electronic unit of the set of M first-level electronic units, calculate a pair of measured positions based on the acquired energy and position readout signals,
use the calculated pairs of measured positions to identify the up to three involved light-sharing segments, based on a particular look-up table from a pre-prepared set of look-up tables, each look-up table corresponding to a probable segment-combination of up to three light-sharing segments within which energy is deposited by gamma-ray interactions occurring during a gamma-ray detection event,
determine a signal level at each photosensor within the identified up to three involved light-sharing segments, based on the calculated pairs of measured positions, peak positions in the particular look-up table that represent positions of corresponding single-light-sharing-segment gamma-ray detection events, and the acquired energy readout signals, and
determine the set of energy, position, and timing signals, based on the determined signal levels at the photosensors within the identified up to three involved light-sharing segments and the acquired energy and timing readout signals.

5. The medical imaging apparatus of claim 4, wherein the second-level electronic unit is further configured to, during a calibration phase:
upon the set of M first-level electronic units being triggered by each gamma-ray detection event of a plurality of gamma-ray detection events, collect the timing, energy, and position readout signals from the set of M first-level electronic units,
generate a flood histogram for each first-level electronic unit of the set of M first-level electronic units, the flood histogram representing a 2D position map of the plurality of gamma-ray detection events detected by the first-level electronic unit,
generate a set of look-up tables for each first-level electronic unit of the set of M first-level electronic units, based on the flood histogram generated for the first-level electronic unit, and
save the generated sets of look-up tables, as the pre-prepared set of look-up tables.

6. The medical imaging apparatus of claim 4, wherein the second-level electronic unit is further configured to determine the set of energy, position, and timing signals by
performing Anger logic calculations based on the determined signal levels at the photosensors within the identified up to three involved light-sharing segments to determine the energy and position signals, and performing weighting calculation based on the acquired energy and timing readout signals to determine the timing signal, or
inputting the determined signal levels at the photosensors within the identified up to three involved light-sharing segments and the acquired energy and timing readout signals into a neural network, and obtaining an output of the neural network as the determined set of energy, position, and timing signals, where the neural network has been trained during a calibration phase to establish a mapping relationship from signal levels at photosensors within up to three light-sharing segments involved in a gamma-ray detection event and energy and timing readout signals acquired for the gamma-ray detection event to energy, position, and timing signals with respect to gamma-ray interactions occurring during the gamma-ray detection event.

7. The medical imaging apparatus of claim 4, wherein the second-level electronic unit is further configured to identify the up to three involved light-sharing segments by:
for each first-level electronic unit of the set of M first-level electronic units, with respect to a voting region with a size dependent on the acquired energy readout signal, casting votes for the pre-prepared set of look-up tables, based on the calculated pair of measured positions,
selecting, as the particular look-up table, a specific look-up table from the pre-prepared set of look-up tables that receives a most number of votes from the set of M first-level electronic units, and
determining, the up to three light-sharing segments included in the segment-combination corresponding to the specific look-up table, as the identified up to three involved light-sharing segments.

8. The medical imaging apparatus of claim 7, wherein each look-up table of the pre-prepared look-up tables has a probability ranking that represents likelihood of the corresponding segment-combination among all of the probable segment-combinations, and the second-level electronic unit is further configured to:
when two or more look-up tables receive the most number of votes from the set of M first-level electronic units, select, based on the respective probability rankings, one of the two or more look-up tables that has higher likelihood, as the particular look-up table,
when two or more look-up tables receive the most number of votes from the set of M first-level electronic units, have a same probability ranking, and correspond to two-light-sharing-segment-combinations, select one of the two or more look-up tables that a vector connecting peak positions representing single-light-sharing segment gamma-ray detection events corresponding to the two light-sharing segments has a better match with a vector representing a linear fit to the measured positions, as the particular look-up table, and
when two or more look-up tables receive the most number of votes from the set of M first-level electronic units, have a same probability ranking, and correspond to three-light-sharing-segment-combinations, and the two three-light-sharing-segment-combinations have a common light-sharing segment, select one of the two or more look-up tables that has more number of votes from the set of M first-level electronic units that do not vote for a look-up table corresponding to a single-light-sharing-segment-combination of the common light-sharing segment, as the particular look-up table.

9. The medical imaging apparatus of claim 2, wherein the particular light-sharing segment includes up to three involved light-sharing segments within which energy was deposited by the gamma-ray interactions occurring during the gamma-ray detection event, and the second-level electronic unit is further configured to:
input the acquired energy and position readout signals into a neural network, and
obtain, at an output of the neural network, segment identifiers of the up to three involved light-sharing segments and a signal level at each photosensor within the up to three involved light-sharing segments, where the neural network has been trained during a calibration phase to establish a mapping relationship from (1) energy and position readout signals acquired for a gamma-ray detection event to (2) up to three light-sharing segments involved in the gamma-ray detection event and signal levels at photosensors within the up to three light-sharing segments involved in the gamma-ray detection event.

10. The medical imaging apparatus of claim 1, wherein each detector module is divided into 16 light-sharing segments, and each light-sharing segment covers 4 photosensors of the photosensor array, or
each detector module is divided into 36 light-sharing segments, and each light-sharing segment covers 9 photosensors of the photosensor array.

11. A method for reading and processing electric signals in a medical imaging apparatus for gamma-ray detection, the medical imaging apparatus including a detector and circuitry, the detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2,
the circuitry including (1) a plurality of sets of M first-level electronic units and (2) a second-level electronic unit, each set of M electronic units being configured to read the electrical signals generated by the photosensor array of a corresponding one of the plurality of detector modules,
the method comprising, for each detector module of the plurality of detector modules:
reading via each first-level electronic unit of a corresponding set of M first-level electronic units, the electrical signals generated by N photosensors in a multiplexing manner to generate timing, energy, and position readout signals on a single set of readout channels; and
processing via the second-level electronic unit, the timing, energy, and position readout signals output from M sets of readout channels of the set of M first-level electronic units, wherein
with respect to each first-level electronic unit of the set of M first-level electronic units, each of the N photosensors read by the first-level electronic unit is situated in a corresponding different one of the N light-sharing segments, and
with respect to each light-sharing segment of the N light-sharing segments, each of the M photosensors covered by the light-sharing segment is read by a corresponding different first-level electronic unit of the set of M first-level electronic units.

12. The method of claim 11, wherein the processing step further comprises:
upon the set of M first-level electronic units being triggered by a gamma-ray detection event, acquiring the timing, energy, and position readout signals from the set of M first-level electronic units,
identifying a particular light-sharing segment within which energy was deposited by gamma-ray interactions occurring during the gamma-ray detection event, based on the acquired energy and position readout signals, and
determining, based on the acquired energy, position, and timing readout signals, a set of energy, position, and timing signals with respect to the gamma-ray interactions occurring during the gamma-ray detection event.

13. The method of claim 12, wherein each light-sharing segment of the N light-sharing segments is configured to encode depth of interaction (DOI) information of gamma-ray interactions occurring therewithin,
the determining step further comprises determining a 3D position signal, as the position signal with respect to the gamma-ray interactions occurring during the gamma-ray detection event, and
the 3D position signal includes position information of the gamma-ray interactions occurring during the gamma-ray detection event, in a depth direction of the scintillation crystals.

14. The method of claim 12, wherein the particular light-sharing segment includes up to three involved light-sharing segments within which energy was deposited by the gamma-ray interactions occurring during the gamma-ray detection event, and the steps of identifying and determining further comprise:
for each first-level electronic unit of the set of M first-level electronic units, calculating a pair of measured positions based on the acquired energy and position readout signals,
using the calculated pairs of measured positions to identify the up to three involved light-sharing segments, based on a particular look-up table from a pre-prepared set of look-up tables, each look-up table corresponding to a probable segment-combination of up to three light-sharing segments within which energy is deposited by gamma-ray interactions occurring during a gamma-ray detection event,
determining a signal level at each photosensor within the identified up to three involved light-sharing segments, based on the calculated pairs of measured positions, peak positions in the particular look-up table that represent positions of corresponding single-light-sharing-segment gamma-ray detection events, and the acquired energy readout signals, and
determining the set of energy, position, and timing signals, based on the determined signal levels at the photosensors within the identified up to three involved light-sharing segments and the acquired energy and timing readout signals.

15. The method of claim 14, further comprises, during a calibration phase:
upon the set of M first-level electronic units being triggered by each gamma-ray detection event of a plurality of gamma-ray detection events, collecting the timing, energy, and position readout signals from the set of M first-level electronic units,
generating a flood histogram for each first-level electronic unit of the set of M first-level electronic units, the flood histogram representing a 2D position map of the plurality of gamma-ray detection events detected by the first-level electronic unit,
generating a set of look-up tables for each first-level electronic unit of the set of M first-level electronic units, based on the flood histogram generated for the first-level electronic unit, and
saving the generated sets of look-up tables, as the pre-prepared set of look-up tables.

16. The method of claim 14, wherein the step of determining the set of energy, position, and timing signals further comprises:
performing Anger logic calculations based on the determined signal levels at the photosensors within the identified up to three involved light-sharing segments to determine the energy and position signals, and performing weighting calculation based on the acquired energy and timing readout signals to determine the timing signal, or
inputting the determined signal levels at the photosensors within the identified up to three involved light-sharing segments and the acquired energy and timing readout signals into a neural network, and obtaining an output of the neural network as the determined set of energy, position, and timing signals, where the neural network has been trained during a calibration phase to establish a mapping relationship from signal levels at photosensors within up to three light-sharing segments involved in a gamma-ray detection event and energy and timing readout signals acquired for the gamma-ray detection event to energy, position, and timing signals with respect to gamma-ray interactions occurring during the gamma-ray detection event.

17. The method of claim 14, wherein the step of using the calculated pairs of measured positions to identify the up to three involved light-sharing segments further comprises:
for each first-level electronic unit of the set of M first-level electronic units, with respect to a voting region with a size dependent on the acquired energy readout signal, casting votes for the pre-prepared set of look-up tables, based on the calculated pair of measured positions,
selecting, as the particular look-up table, a specific look-up table from the pre-prepared set of look-up tables that receives a most number of votes from the set of M first-level electronic units, and
determining, the up to three light-sharing segments included in the segment-combination corresponding to the specific look-up table, as the identified up to three involved light-sharing segments.

18. The method of claim 17, wherein each look-up table of the pre-prepared look-up tables has a probability ranking that represents likelihood of the corresponding segment-combination among all of the probable segment-combinations, and the selecting step further comprises:
when two or more look-up tables receive the most number of votes from the set of M first-level electronic units, selecting, based on the respective probability rankings, one of the two or more look-up tables that has higher likelihood, as the particular look-up table,
when two or more look-up tables receive the most number of votes from the set of M first-level electronic units, have a same probability ranking, and correspond to two-light-sharing-segment-combinations, selecting one of the two or more look-up tables that a vector connecting peak positions representing single-light-sharing segment gamma-ray detection events corresponding to the two light-sharing segments has a better match with a vector representing a linear fit to the measured positions, as the particular look-up table, and
when two or more look-up tables receive the most number of votes from the set of M first-level electronic units, have a same probability ranking, and correspond to three-light-sharing-segment-combinations, and the two three-light-sharing-segment-combinations have a common light-sharing segment, selecting one of the two or more look-up tables that has more number of votes from the set of M first-level electronic units that do not vote for a look-up table corresponding to a single-light-sharing-segment-combination of the common light-sharing segment, as the particular look-up table.

19. The method of claim 12, wherein the particular light-sharing segment includes up to three involved light-sharing segments within which energy was deposited by the gamma-ray interactions occurring during the gamma-ray detection event, and the steps of identifying and determining further comprise:
inputting the acquired energy and position readout signals into a neural network, and
obtaining, at an output of the neural network, segment identifiers of the up to three involved light-sharing segments and a signal level at each photosensor within the up to three involved light-sharing segments, where the neural network has been trained during a calibration phase to establish a mapping relationship from (1) energy and position readout signals acquired for a gamma-ray detection event to (2) up to three light-sharing segments involved in the gamma-ray detection event and signal levels at photosensors within the up to three light-sharing segments involved in the gamma-ray detection event.

20. A non-transitory computer readable medium having instructions stored therein that, when executed by one or more processors, cause the one or more processors to perform a method for reading and processing electric signals in a medical imaging apparatus for gamma-ray detection, the medical imaging apparatus including a detector and circuitry, the detector including a plurality of detector modules, each detector module including a crystal array and a photosensor array coupled to the crystal array, the crystal array including a plurality of scintillation crystals that generate scintillation light in response to gamma-ray interactions occurring therein, the photosensor array including a plurality of photosensors that generate electrical signals upon detecting the scintillation light emitted from the crystal array, each detector module being divided into N light-sharing segments, each of the N light-sharing segments covering M photosensors of the photosensor array, where N and M are integers, N≥2, and M≥2,
the circuitry including (1) a plurality of sets of M first-level electronic units and (2) a second-level electronic unit, each set of M first-level electronic units being configured to read the electrical signals generated by the photosensor array of a corresponding one of the plurality of detector modules,
the method comprising, for each detector module of the plurality of detector modules:
reading via each first-level electronic unit of a corresponding set of M first-level electronic units, the electrical signals generated by N photosensors in a multiplexing manner to generate timing, energy, and position readout signals on a single set of readout channels; and
processing via the second-level electronic unit, the timing, energy, and position readout signals output from M sets of readout channels of the set of M first-level electronic units, wherein
with respect to each first-level electronic unit of the set of M first-level electronic units, each of the N photosensors read by the first-level electronic unit is situated in a corresponding different one of the N light-sharing segments, and
with respect to each light-sharing segment of the N light-sharing segments, each of the M photosensors covered by the light-sharing segment is read by a corresponding different first-level electronic unit of the set of M first-level electronic units.
